SEQUENCE LISTING (49 sequences)

SEQ ID NO: 1 - DNA, 24 bp, Unknown
  primer
actcctccaaactacacccagcaa

SEQ ID NO: 2 - DNA, 26 bp, Trichophyton rubrum
agctctgatctcaccggtgacgatag

SEQ ID NO: 3 - DNA, 22 bp, unknown
  primer
tgccctcagcaagagttgcaat

SEQ ID NO: 4 - DNA, 19 bp, unknown
  primer
gcttggagggctggggagt

SEQ ID NO: 5 - DNA, 20 bp, unknown
  primer
tcactctcgtcgttgctgcc

SEQ ID NO: 6 - DNA, 19 bp, unknown
  primer
cctayggcctcctgatcgt

SEQ ID NO: 7 - DNA, 18 bp, unknown
  primer
ggcagccgtggaggagca

SEQ ID NO: 8 - DNA, 18 bp, unknown
  primer
tggtggactttggtggct

SEQ ID NO: 9 - DNA, 15 bp, unknown
  primer
gcgcycgccrgagga

SEQ ID NO: 10 - DNA, 21 bp, unknown
  primer
ccggaaccaagagatccgttg

SEQ ID NO: 11 - DNA, 24 bp, unknown
  primer
cacattaacttggtcgtyatcggc

SEQ ID NO: 12 - DNA, 18 bp, Unknown
  primer
gaacttctcaatggtacg

SEQ ID NO: 13 - DNA, 32 bp, Unknown
  probe
gccrccgtctctgccgtcactcctccaaacta

SEQ ID NO: 14 - DNA, 28 bp, Unknown
  probe
gagggcaccggcgagtaccagtacagca

SEQ ID NO: 15 - DNA, 25 bp, Unknown
  probe
tccctgcctccctctggcaacccaa

SEQ ID NO: 16 - DNA, 26 bp, Unknown
  probe
caaattcggcgtcaagaacgagggcc

SEQ ID NO: 17 - DNA, 31 bp, Unknown
  probe
cgataccaccgcacttgtagatcaagtgacc

SEQ ID NO: 18 - DNA, 33 bp, Unknown
  probe
gtaatgttatagtcagtttctgtgtaattcggt

SEQ ID NO: 19 - DNA, 768 bp, Trichophyton verrucosum
atgaaggtcactctcgtcgttgctgccctcgcggccgccgtctctgccgtcactcctccaaactactccctgcctccctctggcaaccccatcggcaccccaggcctccacgagaaggtccctgtcgacaagccctacgccatcacctggcaggcaaccaccgagagccatgtctccatcatgctcctccacggctgccccaagaactgcaacccagttcaaactcttgctgagaacatcccgaacaccggcagcctctcttggactcctagttctgacctcaccggtgacgactcctacggcctcgtgatcgtcgtcgagggcaccggccagtaccagtacagcaccaacttcggcatcgagaaccacagccccaagccacagccaccaaagtccaccacgccagccgagaagcctacctggactccccagccctccaagccagtcacccacatcgtcgaggcttcctccagcactcccgtcccctccggcggcgtcatcactctcaccacctccatctgcccgccatctgctaccacttccactgtccccggcgtgccccagccaactggcagcgcgccagtccccggcactcctcacccaaccggcggcaaccctggcccagctccagctccatctggctccggtgctcccgtgccaccaccagcctcgaccaacacccctccaccattcaacaacggtgccggccgcgtcggcgctggcttcggtgccgctctcctcgttgtcgccgctgcttttgccatgtaa

SEQ ID NO: 20 - DNA, 762 bp, Trichophyton tonsurans
atgaagttcactctcgtcgttgctgccttcgcggccaccgtctctgccgtcactcctccaaactacacccagcaaccctccggcaaccccatcactactccgggcctcggcgagcgcgtcccagtcggccaggtcttcaccatctcctggaagccaaccacccagaagcccgtctccatcatgctcctccacggctgcccccagaactgcaacccaattgcaactcttgctgagggcatccccaactccggctctctcccttggactcctgaagctgatctcgtcgatgataacgcctacggcctcctgatcgttgtcgagggcaccggcgagtaccagtacagcacccaattcggcgtcaagaacgacggccccaagccacagccaccaaagtccaccaagccagccgagaagcctacctgggttccccagccctccaagccagtcacccacatcatcgacaccgcctccagcactcccgtcccctccagcggcgtcgtcaccctcaccacctctgtctgcccaccatctgccaccaccgtccccggtgtgccccagccaaccggcagcatgcccatccccggtactcctcagccaactggcggcaacccgggcccagctccagctccgtctggcaccagtgctcccatgccaccaccaggctcgaccaacacccctccaccattcaacaacggtgccggccgcgtcggcgctggtctcggtgccgctttcctcgttctcgccgctgcttttgccatgtaa

SEQ ID NO: 21 - DNA, 795 bp, Trichophyton equinum
atgaagttcactctcgtcgttgctgccttcgcggccaccgtctctgccgtcactcctccaaactacacccagcaaccctccggcaaccccatcactactccgggcctcggcgagcgcgtcacagtcggccaggtcttcaccatctcctggaagccaaccacccagaagcccgtctccatcatgctcctccacggctgcccccagaactgcaacccaattgcaactcttgctgagggcatccccaactccggctctctcccttggactcctgaagctgatctcgtcgatgataacgcctacggcctcctgatcgttgtcgagggcaccggcgagtaccagtacagcacccaattcggcgtcaagaacgacggccccaagccacagccaccaaagtccaccaagccagccgagaagcctacctgggttccccagccctccaagccagtcacccacatcatcgacaccgcctccagcactcccgtcccctccagcggcgtcgtcaccctcaccacctccgtctgcccaccatctgccaccaccgtccccggtgtgccccagccaaccggcagcatgcccatccccggtactcctcagccaaccggcagcgtgcccatccctggcactcagccaactggcggcaacccgggcccagctccagctccgtctggcaccagtgctcccatgccaccaccaggctcgaccaacacccctccaccattcaacaacggtgccggccgcgtcggcgctggtctcggtgccgctttcctcgctctcgccgctgcttttgccatgtaa

SEQ ID NO: 22 - DNA, 798 bp, Trichophyton interdigitale
atgaagttcactctcgtcgttgctgccttcgcggccaccgtctctgccgtcactcctccaaactacacccagcaaccctccggcaaccccatcactgctccgggcctcggcgagcgcgtcccagtcggccaggtcttcaccatctcctggcagccaaccacccagaagcccgtctccctcatgctcctccacggctgccccctgaactgcaacccaattgcaactcttgctgagggcatcccaaactccggctctctcccttggactcctgaagctggtctcgtcgatgatgacgcctacggcctcctgatcgttgtcgagggcaccggcgagtaccagtacagcaccaaattcggcgtcaagaacgagggccccaagccacagccaccaaagtccaccaagccagccgagaagcctacctgggttccccagccctccaagccagtcacccacatcatcgacaccgcctccagcactcccgtcccctccagcggcgtcgtcaccctcaccacctccgtctgcccaccatctgccaccacagtccccggtgtgccccagccaaccggcagcatgcccatccccggtactcctcagccaaccggcagcgtgcccatccctggcactcctcagccaactggcggcaacccgggcccagctccagctccatctggcaccactgctcccatgccaccaccaggctcgaccaacacccctccaccattcaacaacggtgccggccgcgtcggcgctggtctcggtgccgctttcctcgttctcgccgctgcttttgccatgtaa

SEQ ID NO: 23 - DNA, 768 bp, Trichophyton benhamiae
atgaaggtcactctcgtcgttgctgccctcgcggccgccgtctctgccgtcactcctccaaactactcccagcctccctccggcaaccccatcggcaccccaggcctccacgagaaggtccctgtcggccagccctacaccatcacctggcaggcaaccaccgagagccatgtctccatcatgctcctccacggctgccccaagaactgcaacccagttcaaactcttgctgagaacatcccgaactccggcagcctctcttggactcccaagtccgacctcaccggtgacgactcctacggcctcatgatcgtcgtcgagggcaccggccagtaccagtacagcaccaacttcggcatcgagaaccacagccccaagccacagccaccaaagtccaccacgccagtcgagaagcctacctggactccccagccctccaagccagtcacccacatcgtcgagacttcctccagcactcccgtcccctccggcggcgtcatcactctcaccacctccatctgcccgccatctgctaccacttccactgtccccggcgtgccccagccaactggcagcgcgccagtacccggcactcctcacccaaccggcggcaaccctggaccagctccagctccatctggctccggtgctcccgtgccaccaccagcctcgaccaacacccctccaccattcaacaacggtgccggccgcgttggcgctggtttcggtgccgctctcctcgttgtcgccgctgcttttgccatgtaa

SEQ ID NO: 24 - DNA, 759 bp, Trichophyton rubrum
atgaagctcactctcgtcgttgctgccctcgcggccgccgtctctgccgtcactcctccaaactacagccagtctccctccggcaaccccatcgcttccccgggcctccacgagcgcgtccctgtcagcaagccctacaccatcacctggcaggcaaccacctcgagccacgtctccatcatgctcctccacggctgccccaagaactgcgaaccagttgcaacgcttgcggagaacatccccaactccggccaccactcctggactcctagctctgatctcaccggtgacgatagctacggcctcatgattgtcgtcgagggcaccggccagtaccagtacagcaccaacttcggcatcgagaaccacagccccaagccacagccaccaaagtccaccacgccagccgagaagcctacctggactccccagccctccaagccagtcacccatatcatcgagacctccagcactcccgtcccctccggcggcgtcatcactctcaccacctccatctgccctccatccggcaccgccgtccccggtgtgccccatccaaccggcagcgtgcctgtccccggcactcctcacccaaccggcggcaacccaggcccagctccagctccatctggctctggtgctcccatgccaccaccagcctcgaccaacacccctccaccattcaacaacggtgccggccgcgtcggcgctggtctcggtgccgctctcctcgttgtcgccgctgcttttgccatgtaa

SEQ ID NO: 25 - DNA, 759 bp, Trichophyton soudanense
atgaagctcactctcgtcgttgctgccctcgcggccgccgtctctgccgtcactcctccaaactacagccagtctccctccggcaaccccatcgcttccccgggcctccacgagcgcgtccctgtcagcaagccctacaccatcacctggcaggcaaccacctcgagccacgtctccatcatgctcctccacggctgccccaagaactgcgaaccagttgcaacgcttgcggagaacatccccaactccggccaccactcctggactcctagctctgatctcaccggtgacgatagctacggcctcatgattgtcgtcgagggcaccggccagtaccagtacagcaccaacttcggcatcgagaaccacagcccccagccacagccaccaaagtccaccacgccagccgagaagcctacctggactccccagccctccaagccagtcacccatatcatcgagacctccagcactcccgtcccctccggcggcgtcatcactctcaccacctccatctgccctccatccggcaccgccgtccccggtgtgccccatccaaccggcagcgtgcctgtccccggcactcctcacccaaccggcggcaacccaggcccagctccagctccatctggctctggtgctcccatgccaccaccagcctcgaccaacacccctccaccattcaacaacggtgccggccgcgtcggcgctggtctcggtgccgctctcctcgttgtcgccgctgcttttgccatgtaa

SEQ ID NO: 26 - DNA, 759 bp, Trichophyton violaceum
atgaagctcactctcgtcgttgctgccctcgcggccgccgtctctgccgtcactcctccaaactacagccagtctccctccggcaaccccatcgcttccccgggcctccacgagcgcgtccctgtcagcaagccctacaccatcacctggcaggcaaccacctcgagccacgtctccatcatgctcctccacggctgccccaagaactgcgaaccagttgcaacgcttgcggagaacatccccaactccggccaccactcctggactcctagctctgatctcaccggtgacgatagctacggcctcatgattgtcgtcgagggcaccggccagtaccagtacagcaccaatttcggcatcgagaaccacagccccaagccacagccaccaaagtccaccacgccagccgagaagcctacctggactccccagccctccaagccagtcacccatatcatcgagacctccagcactcccgtcccctccggcggcgtcatcactctcaccacctccatctgccctccatccggcaccgccgtccccggtgtgccccatccaaccggcagcgtgcctgtccccggcactcctcacccaaccggcggcaacccaggcccagctccagctccatctggctctggtgctcccatgccaccaccagcctcgaccaacacccctccaccattcaacaacggtgccggccgcgtcggcgctggtctcggtgccgctctcctcgttgtcgccgctgcttttgccatgtaa

SEQ ID NO: 27 - DNA, 617 bp, Trichophyton rubrum
atcattaacgcgcaggccggaggctggccccccacgatagggaccgacgttccatcaggggtgagcagacgtgcgccggccgtacgcccccattcttgtctacctcacccggttgcctcggcgggccgcgctccccctgccagggagagccgtccggcgggccccttctgggagcctcgagccggaccgcgcccgccggaggacagacaccaagaaaaaattctctgaagagctgtcagtctgagcgtttagcaagcacaatcagttaaaactttcaacaacggatctcttggttccggcatcgatgaagaacgcagcgaaatgcgataagtaatgtgaattgcagaattccgtgaatcatcgaatctttgaacgcacattgcgccctctggcattccggggggcatgcctgttcgagcgtcatttcaacccctcaagcccggcttgtgtgatggacgaccgtccggcccctcccttcgggggcgggacgcgcccgaaaagcagtggccaggccgcgattccggcttcctaggcgaatgggcagccaattcagcgccctcaggaccggccgccctggccccaatctttatatatatatatatcttttcaggttgacc

SEQ ID NO: 28 - DNA, 737 bp, Trichophyton rubrum
cacattaacttggtcgttatcggccacgtcgattccggcaaatccaccactactggtaagccagccaccagatacctctagccaggcaccgaattacacagaaactgactataacattacaggtcacttgatctacaagtgcggtggtatcgaccagcgtaccattgagaagttcgagaaggtaaataaccccccttttttgaccctgctgtctccgttctgttgcacaattttcccccttcatcccactacaggtgaaattttggtgctgctggtgggatgtggcttggcactcgcttgggcagcaaaatccacaccccaccaacatcaaacatgcagccatcgctccaggcaacgatcgagcctcatgtcatgtttgggatttgcttttttctaaggatcgatgctaacaaggtacctgtaggaagccgaagagttgggcaagaagtccttcaagtacgcttgggttcttgacaagctcaaggccgagcgtgagcgtggtatcaccatcgatatcgccctctggaagttcgagacccccaagtacaatgtcaccgtcattggtatgtttctttgccttgttccctcatgtggttgtaccatatctaacgagagtagacgcccccggtcaccgtgacttcatcaagaacatgatcactggtacctcccaggctgactgcgctattctcatcattgctgccggtactggtgagttcgaggctggtatctccaaggat

SEQ ID NO: 29 - DNA, 20 bp, Unknown
  PCR target
tcactctcgtcgctgctgcc

SEQ ID NO: 30 - DNA, 20 bp, Unknown
  PCR target
tcactctcgtcgtcgctgcc

SEQ ID NO: 31 - DNA, 32 bp, Unknown
  PCR target
gccgtcgcctctgccgtcactcctccaaacta

SEQ ID NO: 32 - DNA, 25 bp, Unknown
  PCR target
acccagcaaccctccggcaacccca

SEQ ID NO: 33 - DNA, 25 bp, Unknown
  PCR target
tcccagcaaccctccggcaacccca

SEQ ID NO: 34 - DNA, 25 bp, Unknown
  PCR target
gcccagcaaccctccggcaacccca

SEQ ID NO: 35 - DNA, 25 bp, Unknown
  PCR target
agccaggatccctccggcaacccct

SEQ ID NO: 36 - DNA, 25 bp, Unknown
  PCR target
tcccagcctccctccggcaacccca

SEQ ID NO: 37 - DNA, 25 bp, Unknown
  PCR target
tccctgcctccctctggcaacccca

SEQ ID NO: 38 - DNA, 25 bp, Unknown
  PCR target
agccagtctccctccggcaacccca

SEQ ID NO: 39 - DNA, 25 bp, Unknown
  PCR target
tcccagtcgccatccggcaacccca

SEQ ID NO: 40 - DNA, 25 bp, Unknown
  PCR target
tccaactctccatccggcaacccca

SEQ ID NO: 41 - DNA, 25 bp, Unknown
  PCR target
tccaactcgccctccggcaacccca

SEQ ID NO: 42 - DNA, 19 bp, Unknown
  PCR target
cctacggcctcgtgatcgt

SEQ ID NO: 43 - DNA, 19 bp, Unknown
  PCR target
cctacggcctcatgatcgt

SEQ ID NO: 44 - DNA, 19 bp, Unknown
  PCR target
gctacggcctcatgattgt

SEQ ID NO: 45 - DNA, 28 bp, Unknown
  PCR target
gagggcaccggccagtaccagtacagca

SEQ ID NO: 46 - DNA, 26 bp, Unknown
  PCR target
ccaattcggcgtcaagaacgagggcc

SEQ ID NO: 47 - DNA, 26 bp, Unknown
  PCR target
caacttcggcatcgagaacaacagcc

SEQ ID NO: 48 - DNA, 26 bp, Unknown
  PCR target
caacttcggcgtcgagaaccacagcc

SEQ ID NO: 49 - DNA, 26 bp, Unknown
  PCR target
caatttcggcatcgagaaccacagcc

(19) United States
(12) Patent Application Publication
(10) Pub. No.: US 2021/0087641 A1
(43) Pub. Date: Mar. 25, 2021

(54) METHOD FOR DETERMINING DERMATOPHYTES

(71) Applicant: CHARITE - UNIVERSITATSMEDIZIN BERLIN, Berlin (DE)

(72) Inventors: Christiane KUPSCH, Berlin (DE); Yvonne GRASER, Hangelsberg (DE)

(73) Assignee: CHARITE - UNIVERSITATSMEDIZIN BERLIN, Berlin (DE)

(21) Appl. No.: 16/971,163

(22) PCT Filed: Feb. 19, 2019
(86) PCT No.: PCT/EP2019/054035

§ 371 Date: Aug. 19, 2020

(30) Foreign Application Priority Data
Feb. 19, 2018 (EP) 18157464.1

(51) Int. Cl. C12Q 1/6895

(52) U.S. Cl. CPC C12Q 1/6895; C12Q 2600/158

(57) ABSTRACT

The invention relates to methods assessing nucleic acids encoding a dermatophyte extracellular serine/threonine-rich protein (ESTRP gene) in identifying and preferably differentiating between dermatophytes. The invention relates further to corresponding detection kits, in addition to isolated probes and oligonucleotides.

The invention relates to methods assessing nucleic acids encoding a dermatophyte extracellular serine/threonine-rich protein (ESTRP gene) in identifying and preferably differentiating between dermatophytes. The invention relates further to corresponding detection kits, in addition to isolated probes and oligonucleotides.

### BACKGROUND OF THE INVENTION

Dermatophytoses are a special case of dermatomycoses and represent a group of infections of the skin and their appendages (nails and hair) caused by dermatophytes. The dermatophyte-derived medical conditions are often termed Tinea and described in more detail by indicating the infected body site, e.g. Tinea unguium (also onychomycosis or nail mycosis), Tinea pedis, Tinea corporis or Tinea capitis.

The prevalence of dermatophytoses, particularly onychomycosis, has been steadily increasing in recent years and has risen for example in the US from 2.18% in 1979 to 13.8% in 2000 (1, 2). The so-called “Achilles Project”, a European study involving 16 countries and carried out among the general population visiting a general practitioner has demonstrated a prevalence of as much as 26% of the general population (3).

There are a number of predisposing factors that increase the risk of Tinea. In addition to a hereditary disposition and associated diseases, such as diabetes and immunodeficiency, other factors such as sports, environmental factors and the demographic development of the population may play a role (4). In industrialized countries, approximately 20% of the population older than 60 years, and even 50% of the population older than 70 years, suffer from athlete's foot disease (5, 6). In about 20 years, people older than 60 will account for 15-35% of the total population (7). Within a single year (1989-90), the treatment of onychomycosis in the US alone cost $43 million (8). In 1999, $250 million were spent alone on treating mycotic nails (9). In light of the prevalence of tinea infections, in particular in ever-increasing older populations, advances in the diagnosis and treatment of such infections are urgently required.

Dermatophytes are keratinophilic filamentous fungi which infect the skin and its appendages (hairs, nails) of mammals and which are inevitably pathogenic to humans. Essentially every incidence of such a pathogen requires medical treatment.

The approximately 40 dermatophyte species are classified into 7 genera, with only about 20 pathogens from 4 genera, Epidermophyton, Microsporum, Trichophyton and Nannizzia being clinically relevant. The dermatophytes are divided into 3 groups: anthropophilic, zoophilic and geophilic.

Anthropophilic pathogens such as Trichophyton rubrum, T. interdigitale or Epidermophyton floccosum are exclusively transmitted from person to person. Although they can survive in the environment through the formation of chlamydospores for a longer period of time (up to several years), they cannot multiply. On animals, anthropophilic species are found only in exceptional cases, and are typically not infectious in this setting. Thus, humans form the only known ecological niche for this species. These pathogens have adapted to humans and this is the main reason why they usually cause only mild but chronic infections (about 70% of all dermatophytoses) (10).

Zoophilic dermatophyte species are adapted to particular mammalian species and are often specific to a particular mammal. This circumstance is important in the search for the source of infection, because zoophilic species have the potential to be transmitted to humans and cause cutaneous infections (about 30%). The most common sources of infectious agents are pets such as cats (Microsporum canis), guinea pigs (T. benhamiae) and cattle (T. verrucosum), with whom humans have close contact.

The geophilic species are soil inhabitants that live on non-living, keratin-containing substrates. The facultative geophilic species such as Nannizzia gypsea or N. fulva infect animals or humans only very rarely (about 3%) (10), the remaining geophilic species even more rarely.

The two last-mentioned ecological groups cause acute inflammatory dermatophytoses in humans with intact immune systems, since they are not adapted to humans, who therefore represent an accidental host.

For the successful treatment of dermatophytosis, a correct and rapid diagnosis is essential. It can be assumed that a diagnosis based on clinical symptoms alone leads to misdiagnosis in about 50% of all cases (11). The laboratory-based diagnoses are currently based on the direct microscopic detection of fungi and/or the cultivation of pathogen from clinical material. The culture-grown fungal colonies are macroscopically assessed for shape and colour and then microscopically assessed to differentiate the species based on size and shape. A major disadvantage is that conidia, which are essential for species identification, are rarely expressed, especially in the anthrophophilic dermatophyte species. In addition, the cultivation of the usually slow-growing dermatophytes is tedious and takes between two and six weeks to complete. Although direct microscopy can be performed immediately after sampling, it is non-specific; it merely gives an indication of the presence of fungi in general.

In onychomycosis, 30-50% of the microscopically positive findings are not culturable (for example, by self-medication of the patients before a visit to the doctor) and the pathogen is therefore not identifiable at the species level. This suggests a false negative result. The result is that the treatment is discontinued or interrupted for 10 days, the existing fungal cells recover and the pathogen identification can only then be performed once the infection progresses. This uncertainty in conventional diagnostics means that 40% of dermatologists in Germany start oral therapy without actually determining the cause of the disease (12). From a medical, economic and legal point of view, such use of systemic antimycotics should, due to their potential side effects and interactions with the environment, be avoided (13-15).

Species-specific diagnostics are required for additional reasons, for example Microsporum and Trichophyton species respond differently to the common antimycotic classes. The same applies to the therapy duration. Depending on whether anthropophilic or zoophilic pathogens cause the infection, they must be treated for different lengths of time. In addition, a correct species diagnosis can be used to draw conclusions about the animal from which an infection was transferred (usually domestic animals) and this must be treated in order to interrupt the infection chain. A method that identifies the pathogen directly from the clinical material (without any form of precultivation, as is required for mass spectrometry-based approaches, e.g. MALDI-TOF-MS) and is suitable for therapy choice, stratification and control is preferred.

With the introduction of PCR as a microbiological detection method, a tool exists that combines all these advantages, and with which dermatophytes can be differentiated from one another to a species level, typically within 24-48 hours.

For the differentiation of the majority of fungi, the ITS (internal transcribed spacer) regions (ITS1 and ITS2) of the ribosomal DNA are in principle sufficiently variable (16). Universal primers can be designed for the highly conserved, flanking rDNA genes (18S, 5,8S and 28S), while the intervening variable ITS regions are used for identification by probe down to species level (17). However, for the phylogenetically closely related group of dermatophytes, this target DNA region is only of limited suitability since there are only very few polymorphisms between phylogenetically closely related species. Although dermatophyte species can be differentiated by sequencing the entire ITS region, the use of probe-based techniques that are better suited for routine diagnostics is still limited by sequence similarities in the ITS1 and 2 regions of many dermatophytes.

On this basis, PCR approaches have recently been developed and published (18-21). WO 2006/133701 A2 (Statens Serum Institut) teaches a method for detecting dermatophytes in a sample on the basis of PCR primers that amplify a section of the chitin synthase gene that is common to all dermatophytes. Similar approaches are described by amplifying a section from the Internal Transcribed Spacer regions ITS1 and ITS2 (18). A review of dermatophyte diagnostics-performance of different molecular tests and culture regarding the detection of Trichophyton rubrum and Trichophyton interdigitale is provided in (25). Furthermore, a number of commercial test systems are available. However, none of these methods is able to cover a sufficient number of clinically relevant species.

### SUMMARY OF THE INVENTION

In light of the prior art the technical problem underlying the present invention is to provide alternative and/or improved means for identifying one or more dermatophytes in a sample. Another problem underlying the present invention is to provide means for differentiating between dermatophytes. The method of the present invention seeks to provide species specific determination of one or more dermatophytes in a sample, thereby enabling improved treatment stratification and patient management with respect to electing an appropriate treatment regime.

This problem is solved by the features of the independent claims. Preferred embodiments of the present invention are provided by the dependent claims.

The invention relates to the use of a nucleic acid encoding a dermatophyte extracellular serine/threonine-rich protein (ESTRP gene) in identifying and preferably differentiating between dermatophytes.

The invention therefore relates to a method for identifying one or more dermatophytes or nucleic acids thereof, comprising carrying out a nucleic acid amplification reaction on a sample suspected of comprising one or more dermatophytes and/or nucleic acids thereof, wherein said reaction comprises primers that hybridize with a nucleic acid molecule encoding a dermatophyte extracellular serine/threonine-rich protein (ESTRP gene), and assessing the product of the amplification reaction.

To the knowledge of the inventors, there has been no previous disclosure of the novel marker gene ESTRP, as described herein. Although there are various prior art disclosures for PCR-based dermatophyte diagnostics, these do not concern the comprehensive detection of pathogenic dermatophytes or the use of ESTRP, but typically rely only on genus-specific evidence within the ITS region (for example, U.S. Ser. No. 15/222,652 A1, which is hereby incorporated by reference), or enable detection of individual species or complexes, but without extensive coverage of multiple potential pathogens.

None of the commercially available test systems meet the required criteria for comprehensive dermatophyte diagnostics.

(i) Differentiation between the second most common dermatophytosis pathogen T. interdigitale from T. mentagrophytes and differentiation between the anthropophilic species T. tonsurans of the zoophilic species from T. equinum, which differs within the ITS only by a single polymorphism, is not possible. In order to find the source of infection in the case of zoophilic pathogens and to be able to respond to it both therapeutically and preventively, differentiation must be carried out, in order to treat infected animals and to eliminate sources of infection. In anthropophilic species, an accurate diagnosis can disrupt chains of infection and prevent epidemics (23).

(ii) The most prevalent zoophilic dermatophyte in Germany T. benhamiae, which causes highly inflammatory infections, especially in children by contact with guinea pigs (24), cannot be identified by any of the available test systems. Only one kit is able to detect it in complex, but with two other species. At present, no kit known to the inventors is capable of detecting specifically T. benhamiae.

The present invention therefore represents a surprising and beneficial advance in dermatophyte detection and diagnostics. The use of the ESTRP gene in dermatophyte detection has been neither disclosed nor suggested. In preferred embodiments, the present invention enables differentiation between T. interdigitale from T. mentagrophytes, and/or differentiation between T. benhamiae, T. verrucosum and T. erinacei. Differentiation between T. tonsurans and T. equinum is preferably carried out by the use of the ESTRP gene combined with the translation elongation factor 1-α gene (EF-1-alpha gene).

Another weakness of the available test systems of the prior art is the lack of differentiation of rare dermatophytes such as, M. ferrugineum, T. simii or T. concentricum. These pathogens are endemic to Africa, Asia or South America, but may show an increasing prevalence in Europe due to increased migration. It is also conceivable that climatic changes of higher temperatures and humidity will lead to an increased spread of these pathogens. In order to ensure the efficient treatment of such infections and targeted preventive measures, such epidemiological changes must be promptly noted. Accurate and rapid diagnosis should therefore be able to detect all human-pathogenic dermatophytes at the species level.

The present invention, based on the use of the ESTRP gene, optionally combined with the EF-1-alpha gene, in dermatophyte detection, enables detection and differentiation between as T. simii and M. ferrugineum or T. concentricum, which has not been previously thought possible employing established molecular techniques.

In the genome-wide search for new diagnostic markers, the inventors identified the gene ESTRP (extracellular serine/threonine-rich protein), which allows differentiation between many of the relevant dermatophyte species. This gene contains many sequence differences, especially between species whose differentiation has pushed previous molecular diagnostics to their limits. This is particularly clear when looking at the species T. interdigitale, which is the second most common cause of onychomycosis and Tinea pedis worldwide, and T. mentagrophytes, a likewise widespread zoophilic dermatophyte. The gene ESTRP encodes an extracellular serine/threonine-rich protein and has not previously been disclosed as a diagnostic marker.

In preferred embodiments of the invention, the assessment of dermatophyte ESTRP genes employing the methods described herein enables differentiation between the species T. interdigitale and T. mentagrophytes.

In other embodiments, the present invention is designed to (i) expand the pathogen spectrum to be detected to cover all pathogenic dermatophyte species, (ii) include universal dermatophyte detection that minimizes the risk of false negative results, and (iii) includes multiple targets, including the novel ESTRP gene for the reliable species differentiation, even between previously non-differentiable species such as T. interdigitale and T. mentagrophytes.

In one embodiment of the invention, assessing the product of the nucleic acid amplification reaction comprises a melting curve analysis. Through a melting curve analysis, the ESTRP gene sequence amplified by the nucleic acid amplification reaction can be determined in a species-specific manner. In a preferred embodiment, the assessing of the product of the nucleic acid amplification reaction comprises differentiating an identified dermatophyte from other dermatophyte species, wherein a unique melting temperature is assigned for the product of the nucleic acid amplification reaction for one or more of multiple dermatophyte species.

Embodiments of the present invention employing a melting curve analysis enable an accurate and previously impossible molecular determination and/or differentiation of pathogenic dermatophyte species in any given sample, thereby potentially enabling diagnosis and treatment accordingly. A melting curve analysis is associated with the additional advantage of being straightforward in its execution and typically is associated with reduced cost, due to the absence of or significantly reduced amount of fluorescently labelled probes compared with other probe-based assays, as in contrast to other probe-based techniques in a melting curve analysis one single probe is sufficient for the detection and/or differentiation of more than one species.

In one embodiment of the invention, the nucleic acid amplification reaction is a quantitative real-time polymerase chain reaction (qRT-PCR).

Quantitative RT-PCR enables the fast, accurate and reliable analysis with low risks of contamination. In principle, two PCR methods may be used for routine microbiological diagnostics. These include classical end-point PCRs, which require a post-procedure for species differentiation by probe hybridization, for example in the form of a PCR ELISA, a blot method or a microarray. Classical end-point PCR is not quantitative and takes 8-10 hours due to the post-procedure. Opening the tubes with previously amplified DNA as part of species differentiation can lead to potential contamination. The second method, real-time PCR, on the other hand, is preferred in the present invention and is low in contamination because both DNA replication and species differentiation by probe hybridization occur in the same tube. This makes the method faster (1-2 h) and it is also able to quantify the infectious load.

In one embodiment, the method comprises a melting curve analysis using a sequence-unspecific double-stranded DNA binding dye, and/or one or more labelled sequence-specific probes that hybridizes to the ESTRP gene. In some embodiments, the invention comprises both a melting curve analysis using a sequence-unspecific double-stranded DNA binding dye and one or more labelled sequence-specific probes that hybridizes to the ESTRP gene.

In one embodiment, the method comprises determining the presence of and/or differentiating between one or more species of the genera Trichophyton, Epidermophyton, Microsporum and/or Nannizzia.

In a preferred embodiment, the invention comprises identification of and/or differentiation between one or more of T. interdigitale, T. tonsurans, T. equinum, T. soudanense, T. violaceum, T. rubrum, T. benhamiae and T. verrucosum.

In a preferred embodiment, the invention comprises differentiation between T. interdigitale from T. mentagrophytes.

In a preferred embodiment, the invention comprises differentiation between T. tonsurans from T. equinum.

In a preferred embodiment, the invention comprises differentiation between T. benhamiae, T. concentricum, T. verrucosum and T. erinacei.

The embodiments mentioned above represent a non-limiting list of dermatophytes that can be assessed using the method of the present invention. A skilled person is capable of determining sequence differences between said species and means for assessing these differences using established molecular biological techniques, such as those disclosed herein.

In a preferred embodiment of the invention, the primers that hybridize with the ESTRP gene are characterized in that:
a. the ESTRP gene sequence bound by the primers exhibits three or fewer, preferably two or fewer, nucleotide differences in dermatophyte species within the genera Trichophyton, Epidermophyton and Microsporum, and/or species of the genus Nannizzia (enabling amplification of the ESTRP gene sequence of any one or more of said species), and b. the ESTRP gene sequence between the sequences bound by the primers and amplified by the nucleic acid amplification reaction exhibits sufficient sequence diversion between one or more of dermatophyte species within the genera Trichophyton, Epidermophyton, Microsporum (preferably determining a species Microsporum canis, M. ferrugineum) and/or Nannizzia (preferably determining a species Nannizzia gypsea, N. fulva, N. incurvata and N. persicolor) to enable unique melting temperatures in a melting curve analysis for one or more of said dermatophyte species and/or genera.

Through the combination of the above features a. and b., the amplification of a dermatophyte ESTRP gene from a dermatophyte species within the genera Trichophyton, Epidermophyton, Microsporum and/or Nannizzia is possible, due to (a.) the primers exhibiting sufficient sequence identity with the corresponding target sequences in order to amplify a region of a ESTRP gene. Subsequently a melting curve analysis is capable of determining dermatophyte species within the genera Trichophyton, Epidermophyton, Microsporum (preferably determining a species Microsporum canis, M. ferrugineum) and/or Nannizzia (preferably determining a species Nannizzia gypsea, N. fulva, N. incurvata and N. persicolor), due to (b.) the amplified region exhibiting sufficient sequence differences to provide a distinct melting temperature.

The definition of the region of the ESTRP gene to be amplified according to a. and b. above can be considered in structural and functional terms as sufficient for a skilled person to identify regions of the ESTRP gene capable of enabling the present method. Methods for determining sequence identities between various regions of ESTRP encoding regions, for calculating estimated melting temperatures and for determining melting temperatures by experimental approaches are known to a skilled person and are described in more detail below. The functional definition of an amplified region exhibiting sufficient sequence difference to another sequence to provide a distinct melting temperature relates directly into clear structural features based on the sequences to be compared. A skilled person is capable of determining sequence differences between said species and means for assessing these differences via melting temperature using established molecular biological techniques, such as those disclosed herein.

By way of example, disclosed herein are sequences of ESTRP encoding nucleic acids of Trichophyton verrucosum, Trichophyton tonsurans, Trichophyton equinum, Trichophyton interdigitale, Trichophyton rubrum, Trichophyton soudanense and Trichophyton violaceum (SEQ ID NO 19-26). With alignments of these sequences (as in FIG. 8) and of ESTRP encoding nucleic acids of other dermatophyte species, a skilled person is capable of determining regions of such nucleic acids against which primers may be designed, and in which distinct melting temperatures may be obtained.

Additional sequencing of other dermatophyte species can be carried out and appropriate regions of the ESTRP gene and/or other genomic regions conducted, in order to identify regions suitable for primer or probe binding, in addition to regions sufficient for differential melting temperature analysis.

As described below in FIGS. 4-7, the ESTRP gene sequences of T. tonsurans, T. equinum, T. interdigitale, T. mentagrophytes, T. quinckeanum, T. schoenleinii, T. simii, T. erinacei, T. concentricum, T. benhamiae, T. verrucosum, T. rubrum, T. soudanense, T. violaceum, E. floccosum, N. persicolor, N. gypsea, N. fulva, N. incurvata, M. canis, and M. ferrugineum have been determined and compared for sequence identities. On the basis of sequencing these and other dermatophyte species suitable gene regions may be determined for designing primer and/or probe target sequences for use in the method of the present invention.

Examples of target regions for primers and probes in the ESTRP gene for multiple dermatophyte species are provided in FIG. 2, which shows alignments of target sequences (for probes and primers) of particular, but non-limiting, regions of the ESTRP gene (regions ESTRP-I and ESTRP-II; SEQ ID NO 29-49). By way of example, on the basis of such target sequences, primers have been designed that show conservation across multiple species, and in combination with target regions for probes that show variation across species, determination of and/or differentiation between one or more of the above-mentioned species is possible.

In a preferred embodiment of the present invention, the method comprises:
a. a first qRT-PCR reaction, wherein said first reaction comprises primers that hybridize with a dermatophyte ESTRP gene, and wherein the first reaction (product) is assessed using a melting curve analysis with a sequence-unspecific double-stranded DNA binding dye, and
b. a second qRT-PCR reaction, wherein said second reaction comprises primers that hybridize with a dermatophyte ESTRP gene, and wherein the second reaction (product) is assessed using a melting curve analysis with one or more labelled sequence-specific probes that hybridize to the ESTRP gene.

The combined use of both a melting curve analysis of a first PCR product with a sequence-unspecific double-stranded DNA binding dye with a second PCR and assessment using a melting curve analysis with one or more labelled sequence-specific probes that hybridize to the ESTRP gene, and optionally to other DNA markers in a multi-locus reaction, provides coverage of a broad range of dermatophytes, thereby essentially enabling detection of any given pathogenic strain.

In a preferred embodiment of the method, the second reaction comprises a first and a second labelled sequence-specific probe that hybridize to the ESTRP gene, wherein
a. the first probe (anchor probe) is 15 to 40 nucleotides in length and hybridizes to a conserved sequence of the dermatophyte ESTRP gene with three or fewer, preferably two or fewer, nucleotide differences in the ESTRP gene sequence in dermatophyte species within the genera Trichophyton, Epidermophyton and Microsporum, and/or species of the genus Nannizzia (preferably Nannizzia gypsea, N. fulva N. incurvata and N. persicolor), and
b. the second probe (species-specific probe) is 15 to 40 nucleotides in length and hybridizes to a sequence of the dermatophyte ESTRP gene with sufficient sequence diversion between one or more of the species within the genera Trichophyton, Epidermophyton and Microsporum (preferably M. canis and M. ferrugineum), and/or species of the genus Nannizzia (preferably Nannizzia gypsea, N. fulva, N. incurvata and N. persicolor) to enable unique melting temperatures in a melting curve analysis for said second probe for one or more of said dermatophyte species and/or genera, and
c. said first and second probes hybridize in proximity to each other on the ESTRP gene and comprise labels enabling fluorescence resonance energy transfer (FRET) when in physical proximity.

According to the present invention, a “sequence specific probe” or “species probe” is preferably, but must not necessarily be, unique for only one species to be identified. In some cases, the “species probe” will potentially bind to a target sequence of two or more species, and show separation from said target sequence at a similar temperature.

The definition of the region of the ESTRP gene to be bound by the probes of a., b. and c. above can be considered in structural and functional terms as sufficient for a skilled person to identify regions of the ESTRP gene capable of enabling the present method. Methods for determining sequence identities between various regions of ESTRP encoding regions, for calculating melting temperatures and for determining the distance between anchor and species-specific probe for functional interaction are known to a skilled person and are described in more detail below.

In a preferred embodiment, the primers that hybridize with the ESTRP gene bind to and amplify a region of the ESTRP gene positioned between nucleotides 1 and 250 and/or between nucleotides 250 and 470 and/or between nucleotides 470 and 768, with reference to the ESTRP gene from T. verrucosum strain HKI 0517 (SEQ ID NO 19). These regions enable a beneficial combination of sequence conservation for primer binding to enable amplification of ESTRP gene sequences from multiple dermatophytes, in combination with variable internal regions for differential melting temperatures.

In a preferred embodiment, the primers for the first reaction comprise or consist of a sequence according to one or more of SEQ ID NO 1 and/or 2 as forward primers and SEQ ID NO 3 and/or 4 as reverse primers. In other embodiments, functionally similar oligonucleotides may be employed with variable sufficient sequence identity.

In a preferred embodiment, the primers for the second reaction comprise or consist a sequence according to one or more of SEQ ID NO 5 and/or 6 as forward primers and SEQ ID NO 7 and/or 8 as reverse primers.

In a preferred embodiment, the one or more sequence-specific probes of the second reaction bind to a region of the ESTRP gene between nucleotides 70 and 110 and/or between nucleotides 360 and 410, with reference to the ESTRP gene from T. verrucosum strain HKI 0517 (SEQ ID NO 19). These regions enable a beneficial combination of sequence conservation for primer binding to enable amplification of ESTRP gene sequences from multiple dermatophytes, in combination with variable internal regions for differential melting temperatures.

In a preferred embodiment, the probes comprise or consist of a sequence according to one or more of SEQ ID NO 13 and/or 14 as anchor probes and SEQ ID NO 15 and/or 16 as species specific probes.

In one embodiment of the invention, the method is characterized in that the first reaction comprises additionally primers that hybridize with a dermatophyte internal transcribed spacer region 1 and/or 2 (ITS1 and/or ITS2 region).

In a preferred embodiment, the primers that hybridize with the ITS1 and/or ITS2 region bind to and amplify a region of the ITS1 and/or ITS2 region between nucleotides 150 and 350, with reference to the ITS1 and/or ITS2 region from T. rubrum (SEQ ID NO 27).

In a preferred embodiment, the primers comprise or consist of a sequence according to SEQ ID NO 9 as forward primer and SEQ ID NO 10 as reverse primer.

In one embodiment of the invention, the method is characterized in that the second reaction comprises additionally primers that hybridize with a dermatophyte translation elongation factor 1-α gene (EF-1-alpha gene).

In one embodiment of the invention, the primers that hybridize with the EF-1-alpha gene bind to and amplify a region of the EF-1-alpha gene between nucleotides 1 and 230, with reference to the EF-1-alpha gene from T. rubrum (SEQ ID NO 28).

In one embodiment of the invention, the primers comprise or consist of a sequence according to SEQ ID NO 11 as forward primer and SEQ ID NO 12 as reverse primer.

In one embodiment of the invention, the one or more sequence-specific probes are employed in the second reaction and bind to a region of the EF-1-alpha gene, preferably wherein the probes comprise or consist of a sequence according to one or more of SEQ ID NO 17 as anchor probe and SEQ ID NO 18 as species specific probe.

This multi-locus approach of the present invention, interrogating ESTRP, ITS regions and/or EF-1-alpha has the advantage that a species is not identified by a single melting temperature, but by multiple melting peaks based on different regions in the genome. Thus, misidentification by mutations (intraspecific variability) is essentially ruled out, as it is extremely unlikely that such mutations will occur at the same time in the same strain at the binding sites of three probes.

With respect to improvements of the present invention over the prior art, in addition to the limited specificity of the target genes used in prior art test systems, a common weakness of probe-based systems is their susceptibility to making false negatives when mutations occur within the chosen target sequence or unusual pathogens are present, that are not detectable through the specific probes employed. This problem can be minimized by a multi-locus analysis as provided in the present invention.

A preferred technique of the present invention is beneficial as it is fast and associated with low risks of contamination. The interpretation of the results is straightforward and a software-supported evaluation is possible of melting temperatures. For species identification, only temperature values need to be read. The present invention is therefore in some embodiments based on a qRT-PCR with a subsequent melting curve analysis, made beneficial by selection of a genome section that is variable enough for differentiation but has conserved sections in the flanking region for primer design and for anchor probe positioning.

The heterogeneous region is needed as a binding site for a species-specific probe and the conserved regions serve as a binding site for the so-called anchor probe.

In preferred embodiments, both probes are fluorescently labelled in such a manner that fluorescence energy transfer (FRET) can take place between them if they are in close proximity to each other. If both probes (anchor and species-specific probe) are bound to their target sequences, then a fluorescence signal can be measured whose wavelength depends on the probe selected. If the temperature is gradually increased, the hydrogen bonds that exist between the probe and the DNA strand are disrupted and the probes separate from the target DNA. Thus, the close proximity of the two probes is no longer existent and there is no FRET signal. The intensity of the fluorescence signal is continuously measured during the temperature increase and the strength of the signal is recorded as a function of the temperature. The temperature at which 50% of the probe molecules have separated from the target DNA is typically designated as the melting temperature (Tm). This temperature depends on the nucleotide sequence of the probe, in particular on whether the probe is 100% complementary to the corresponding binding site on the DNA.

For example, the specific probe preferably fits with 100% sequence identity to the target sequence of a first species, to the target sequence of a second species there is however a polymorphism, i.e. a mismatch, and to a potential third species there may be two mismatches. The detected melting temperature is therefore highest when the first species is present and in each case lower when the second or third species are present in a sample, since in the latter case fewer hydrogen bonds must be disrupted through elevation of the temperature.

Pathogen detection can not only be conducted via fluorescence-labeled probes, but also via dyes that intercalate into double-stranded DNA, such as SybrGreen or EvaGreen. This analysis is not dependent on the separation between a short probe and the target sequence, but the separation of longer double-stranded DNA segments (i.e. SybrGreen is bound to the DNA, leading to the fluorescence signal being measurable) into single strands (i.e. SybrGreen is no longer bound to the DNA and no signal can be measured). This variant is cost-effective, since no fluorescence-labeled probes, which are typically expensive to produce, are needed.

In a preferred embodiment, the invention combines one or more PCR reactions with both one or more fluorescence-labeled probes and one or more sequence-unspecific double-stranded DNA binding dyes.

In a preferred embodiment, the first reaction is to determine whether a dermatophyte is present in a clinical sample. Preferably, this first reaction comprises primers that hybridize with a dermatophyte ESTRP gene, and wherein the first reaction (product) is assessed using a melting curve analysis with a sequence-unspecific double-stranded DNA binding dye.

At the same time, this reaction is able to determine whether a pathogen in the sample is T. rubrum. This applies to approximately 90% of all clinical samples from patients with suspected dermatophytosis (18-20, 26).

This first reaction is preferably based solely on the EvaGreen dye or an equivalent dsDNA binding dye. A further analysis, in a second reaction, typically only need to be carried out for the samples (typically about 10% of all possible samples) in which another dermatophyte species is present.

A second reaction is then carried out with fluorescence-labeled probes.

In a preferred embodiment, employing three different probes (preferably selected from SEQ ID NO 13-18), 18 of the 20 pathogenic dermatophyte species are differentiated. Two species are detected as a complex.

In preferred embodiments, two of these three probes are located in the novel marker gene ESTRP. The third probe is preferably targeted to the translation elongation factor-1-alpha gene, which is already known as a suitable marker for the differentiation of dermatophytes and other pathogenic fungi (27), but has not yet been used for commercial dermatophyte diagnostics.

The combination of both techniques (sequence unspecific dsDNA binding dyes and labelled hybridization probes) enables a cost-saving, species-specific dermatophyte determination or diagnosis of infection that has not been possible before.

A further aspect of the invention relates to a kit for identifying one or more dermatophytes or nucleic acids thereof, comprising one or more reagents for a nucleic acid amplification reaction on a sample suspected of comprising one or more dermatophytes and/or nucleic acids thereof, wherein said reagents comprise (i) primers that hybridize with a ESTRP gene and preferably (ii) software configured for identifying dermatophytes and/or differentiating dermatophytes from other dermatophyte species on the basis of unique melting temperatures assigned to the products of the nucleic acid amplification reaction for one or more multiple dermatophyte species.

The present invention may therefore be realized as a test system or kit that can be employed by diagnostic laboratories and dermatological clinics to include high quality dermatophyte diagnostics. The test system or kit contains preferably all the PCR reagents needed to perform the qRT-PCR and the melting curve analysis and is designed in such a way that as few steps as possible are required to prepare the reaction and the evaluation. In preferred embodiments, there is a ready PCR mix to which only the DNA of the clinical sample needs to be pipetted and a software for evaluation is included that provides a finding automatically, or after entering the obtained melting temperatures.

In a preferred embodiment, the kit of the present invention comprises additionally:
a. One or more sequence-unspecific double-stranded DNA binding dyes and
b. One or more labelled sequence-specific probes that hybridize to a dermatophyte ESTRP gene, preferably probes according to SEQ ID NO 13-16.

A further aspect of the invention relates to an isolated oligonucleotide of 15 to 40 nucleotides in length, comprising or consisting of a sequence according to any one of:
a. SEQ ID NO 1-8, preferably in the form of a primer; or
b. SEQ ID NO 13-16, preferably in the form of a probe.

The invention therefore covers the particular primer and probe sequences as described herein, which are associated with the benefits described in detail above with respect to the method of the invention. The primers and probes of the present invention may also be prepared as a set of oligonucleotides, comprising one or more of the oligonucleotides described herein, in particular the groups of oligonucleotides in the functional combination as described above with respect to the method of the present invention.

The invention further relates to a method for the diagnosis of a dermatophyte infection (dermatophytosis) in a subject, comprising:
a. obtaining, providing and/or preparing a sample obtained from a subject suspected of having said infection,
b. carrying out the method of the invention as described herein, and/or
c. determining the pathogen responsible for said infection.

In preferred embodiments of the invention the particular dermatophyte pathogen may be identified by the present method and distinguished from other potential pathogens. The diagnosis of the dermatophyte infection therefore enables informed decisions regarding appropriate treatment regimes, thereby reducing the administration of unsuitable treatment with antibiotics or insufficient anti-mycotic agents and speeding recovery of the subject from said infection.

In some embodiments, the method comprises the transmission of the diagnosis to the subject from whom the sample was obtained. The diagnosis may be communicated to said subject by the responsible clinician, or clinical laboratory who has conducted the analysis.

The invention further relates to a method for the treatment of a dermatophyte infection (dermatophytosis) in a subject, comprising:
a. obtaining, providing and/or preparing a sample obtained from a subject suspected of having said infection,
b. carrying out the method of the invention as described herein,
c. determining the pathogen responsible for said infection, and
d. administering one or more anti-mycotic agents suitable for the treatment of the pathogen identified.

### DETAILED DESCRIPTION OF THE INVENTION

The invention relates to methods assessing nucleic acids encoding a dermatophyte extracellular serine/threonine-rich protein (ESTRP gene) in identifying and preferably differentiating between dermatophytes. The invention relates further to corresponding detection kits, in addition to isolated probes and oligonucleotides.

According to the present invention, the term “identifying” relates to the determination of the presence of one or more particular dermatophyte genera or species present in a sample. The term “differentiating” an identified dermatophyte from other dermatophyte species relates to providing information on whether the one or more particular genera or species are present, and that one or more particular genera or species are absent in a sample, or to determining that the likelihood is higher, that one or more identified dermatophyte genera or species is present and other genera or species are absent.

The nucleic acid encoding a dermatophyte extracellular serine/threonine-rich protein (ESTRP gene) can be determined by a skilled person using methods established in the field of molecular microbiology, for example by sequence analysis and identification of coding nucleic acids in fungal species of sufficient sequence identity and/or functional analogy or similarity to the nucleic acid sequences of SEQ ID NO 19-26 and the function of corresponding encoded proteins.

The term “assessing” the product of the amplification reaction product encompasses any means of interrogating a nucleic acid amplified product in order to determine its structure or function, including in preferred embodiments sequence analysis via nucleic acid sequencing, a melting curve analysis, for example in order to determine a Tm of the obtained nucleic acid product, analysis by gel electrophoresis, or other methods known to a skilled person.

The terms “polynucleotide”, “nucleotide”, “nucleotide sequence”, “nucleic acid”, “nucleic acid molecule”, “nucleic acid sequence” and “oligonucleotide” may be used interchangeably, and can also include plurals of each respectively depending on the context in which the terms are utilized. They refer to a polymeric form of nucleotides of any length, either deoxyribonucleotides (DNA) or ribonucleotides (RNA), or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA, ribozymes, small interfering RNA, (siRNA), microRNA (miRNA), small nuclear RNA (snRNA), cDNA, recombinant polynucleotides, branched polynucleotides, plasmids, vectors, isolated DNA (A, B and Z structures) of any sequence, PNA, locked nucleic acid (LNA), TNA (treose nucleic acid), isolated RNA of any sequence, nucleic acid probes and primers.

The term “nucleic acid amplification reaction” refers to any method comprising an enzymatic reaction, which allows the amplification of nucleic acids. One preferred embodiment of the invention relates to a polymerase chain reaction (PCR). Another preferred embodiment relates to real time PCR (RT-PCR) or quantitative RT-PCR (qRT-PCR), as it allows the quantification of the amplified target in real-time. The term “real-time PCR” is intended to mean any amplification technique which makes it possible to monitor the progress of an ongoing amplification reaction as it occurs (i.e. in real time). Data is therefore collected during the exponential phase of the PCR reaction, rather than at the end point as in conventional PCR. Measuring the kinetics of the reaction the early phases of PCR provides distinct advantages over traditional PCR detection. In real-time PCR, reactions are characterized by the point in time during cycling when amplification of a target is first detected rather than the amount of target accumulated after a fixed number of cycles. The higher the starting copy number of the nucleic acid target, the sooner a significant increase in fluorescence is observed. Traditional PCR methods may also be applied, and use separation methods, such as agarose gels, for detection of PCR amplification at the final phase of or end-point of the PCR reaction. For qRT-PCR no post-PCR processing of the unknown DNA sample is necessary as the quantification occurs in real-time during the reaction. Post-processing, for example by a melting curve analysis, is also possible. Furthermore, an increase in reporter fluorescent signal is directly proportional to the number of amplicons generated.

Although nucleic acid amplification is often performed by PCR or RT-PCR, other methods exist. Non-limiting examples of such method include quantitative polymerase chain reaction (Q-PCR), ligase chain reaction (LCR), transcription-mediated amplification (TMA), self-sustained sequence replication (3SR), nucleic acid sequence-based amplification (NASBA), strand displacement amplification (SDA), recombinase polymerase amplification (RPA), loop-mediated isothermal amplification (LAMP), helicase-dependent amplification (HDA), helicase-dependent isothermal DNA amplification (tHDA), branched DNA (bDNA), cycling probe technology (CPT), solid phase amplification (SPA), rolling circle amplification technology (RCA), real-time RCA, solid phase RCA, RCA coupled with molecular padlock probe (MPP/RCA), aptamer based RCA (aptamer-RCA), anchored SDA, primer extension preamplification (PEP), degenerate oligonucleotide primed PCR (DOP-PCR), sequence-independent single primer amplification (SISPA), linker-adaptor PCR, nuclease dependent signal amplification (NDSA), ramification amplification (RAM), multiple displacement amplification (MDA), real-time RAM, and whole genome amplification (WGA).

The PCR cycle parameters may be any suitable set of cycle parameters for amplifying the nucleotide sequences targeted by the primers, when the sample contains nucleic acids that include the target nucleotide sequences in detectable amounts. In some embodiments, the cycle parameters include a denaturing temperature in the range of 90 to 100° C., a denaturing time in the range of 5 to 45 seconds; an annealing temperature that may vary with the primers used in the reaction, and may be in the range of 45 to 75° C., and an annealing time of 5 to 45 seconds; and an extension temperature in the range of 60 to 75° C., and an extension time in the range of 20 to 120 seconds. The PCR cycle may include detection of amplification products in the reaction mixture by, e.g., detecting the level of fluorescence in the reaction mixture at the end of a cycle. The number of cycles may range from 18 to 45 cycles, such as 20 to 40 cycles. In certain embodiments, the number of cycles is from 30 cycles to 45 cycles, e.g., from 33 cycles to 38 cycles, including from 35 cycles to 37 cycles. To obtain a melting temperature of amplification products, the PCR protocol may include a melting curve analysis step after the PCR cycles are completed.

A “melting curve analysis” is a known method to the person skilled in the art and is an established method for characterizing amplicons (products of nucleic acid amplification reactions). A melting curve analysis may also be used as an alternative to fluorescent techniques. Melting curve analysis is an assessment of the dissociation-characteristics of double-stranded DNA during heating. As the temperature is raised, the double strand begins to dissociate leading to a rise in the absorbance intensity, hyperchromicity. The information gathered can be used to infer the presence and identity of sequence, for example single-nucleotide polymorphisms (SNP). This is due to the fact that G-C base pairing have 3 hydrogen bonds between them while A-T base pairs have only 2. DNA with a higher G-C content, whether because of its source or, as previously mentioned, because of SNPs, will have a higher melting temperature than DNA with a higher A-T content. Melting curve analysis, for example of PCR products via SYBR Green, other double-strand specific dyes, or probe-based melting curve analysis has become common. The probe-based technique is sensitive enough to detect single-nucleotide polymorphisms (SNP) and can distinguish between homozygous wildtype, heterozygous and homozygous mutant alleles by virtue of the dissociation patterns produced. With higher resolution instruments and advanced dyes, amplicon melting analysis of one base variants is now possible with several commercially available instruments. For example: Applied Biosystems 7500 Fast System and the 7900HT Fast Real-Time PCR System, Idaho Technology's LightScanner (a plate-based high-resolution melting device), Qiagen's Rotor-Gene instruments, and Roche's LightCycler 480 instruments.

Typically, an unspecific dye is incorporated during DNA or cDNA amplification. During the temperature-dependent dissociation of two DNA-strands, the unspecific dye is released and can be detected in a detection channel. The release of unspecific dye directly correlates with the stability and composition of the DNA and allows to scan for sequence variations in an unknown sample. Single-base changes in the target amplicons are detected by their altered melting-properties which is monitored through the release of fluorescent double-stranded DNA binding dye. These altered melting properties give rise to changes in the shape of the melting curve compared to a known sample and allow the characterization of the unknown sample. The preferred method of the invention allows the characterisation of a target by analysing the release of the unspecific dye during melting curve analysis. As the unspecific dye emits light which is distinguishable from the light emitted by the sequence-specific probes, the release of the fluorophore can be used to analyse the composition of the target and identify primer dimers.

Additional methods employing a melting curve analysis are described herein, for example those embodiments in which sequence specific probes are employed. For example, FRET-based approaches are encompassed by the present invention in which one or more probes are labelled with multiple fluorescent labels, for example two probes (preferably anchor and sequence-specific probes) each with a different label, that interact when in physical proximity (i.e. when bound to target nucleic acid sequences) in order to provide a FRET signal. Upon increasing the temperature in a melting curve analysis, the probes are separated from their target sites, thereby disrupting the FRET interaction of the labels leading to a reduction in signal.

In some embodiments of the invention, the assessing of the amplification product may comprise a melting curve analysis, which thereby preferably comprises obtaining one or more melting temperature (Tm) values for reaction products of the real-time PCR performed using the method described herein, or for determining Tm of particular combinations of FRET probes. The temperature at which 50% of the probe molecules have separated from the target DNA is typically designated as the melting temperature (Tm). The method may therefore comprise comparing the obtained Tm values with one or more reference Tm ranges for specific dermatophyte species, wherein the dermatophyte species is determined to be present in the sample when the one of more Tm values is within the one or more reference Tm ranges specific for the dermatophyte species.

In some embodiments of the invention, dermatophyte-specific primers are configured to amplify a dermatophyte ESTRP encoding nucleic acid. In some cases, the dermatophyte-specific nucleic acid products are distinguishable by having distinct expected Tm range(s). Thus, in some cases, the dermatophyte-specific primers may be configured to amplify a dermatophyte-specific ESTRP nucleic acid having different expected Tm ranges, depending on the species from which the ESTRP gene arises. The expected Tm ranges of different species-specific Tm ranges may be different by 0.1° C. or more, 0.5° C. or more, e.g., 1° C. or more, 2° C. or more, 3° C. or more, 4° C. or more, 5° C. or more, 6° C. or more, 8° C. or more, including 10° C. or more, for example, as measured between the medians of the respective ranges.

The term “primer” as used herein refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, i.e., in the presence of nucleotides and an inducing agent such as DNA polymerase and at a suitable temperature and pH. The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and use of the method. For example, for diagnostics applications, depending on the complexity of the target sequence, the oligonucleotide primer typically contains 8 to 50, preferably 12-30, or 15-40, or more nucleotides, although it may contain fewer nucleotides.

The term “probe” relates to a nucleic acid oligonucleotide probe targeting (or binding or hybridizing to) internal regions of the PCR amplicons (products) generated using the amplification primer combinations described herein are encompassed by the present invention. The group of PCR-generated nucleic acid templates is prepared from one or more of the target microbial species mentioned above. These probes can be used for real-time PCR detection (e.g. TaqMan probes, molecular beacons). As used herein, the term “probe” refers to a single-stranded nucleic acid sequence that can be hybridized with a complementary single-stranded target sequence to form a double-stranded molecule (hybrid). The probe is labelled with one or more fluorescent labels or fluorophores. Said oligonucleotide sequence of the probe comprises, consists or essentially consists of 5 to 100 bases, preferably 5 to 50, 10 to 40, 15 to 40, 12 to 38, 13 to 35, 14 to 33, or 15 to 30 bases, more preferably 25-35 bases. The length of the probe is preferably between 15 and 40 nucleotides in length.

A “fluorescent label” or “fluorophore” is a fluorescent chemical compound that can re-emit light upon light excitation. Fluorophores for use as labels in constructing labeled probes of the invention comprise, without claiming to be exhaustive, rhodamine and derivatives, such as Texas Red, fluorescein and derivatives, such as 5-bromomethyl fluorescein, Lucifer Yellow, IAEDANS, 7-Me2N-coumarin-4-acetate, 7-OH-4-CH3-coumarin-3-acetate, 7-NH2-4CH3-coumarin-3-acetate (AMCA), monobromobimane, pyrene trisulfonates, such as Cascade Blue, and monobromotrimethyl-ammoniobimane, FAM, TET, CAL Fluor Gold 540, HEX, JOE, VIC, CAL Fluor Orange 560, Cy3, NED, Quasar 570, Oyster 556, TMR, CAL Fluor Red 590, ROX, LC red 610, CAL Fluor Red 610, Texas red, LC red 610, CAL Fluor Red 610, LC red 640, CAL Fluor Red 635, Cy5, LC red 670, Quasar 670, Oyster 645, LC red 705, Cy5.5, BODIPY FL, Oregon Green 488, Rhodamine Green, Oregon Green 514, Cal Gold, BODIPY R6Gj, Yakima Yellow, JOE, HEX, Cal Orange, BODIPY TMR-X, Quasar-570/Cy3, TAMRA, Rhodamine Red-X, Redmond Red, BODIPY 581/591, Cy3.5, Cal Red/Texas Red, BODIPY TR-X, BODIPY 630/665-X, Pulsar-650, Quasar-670/Cy5.

FRET probes for RT-PCR may be, without limitation: Donor probe 1 (3′ Fluorescein) LC610, LC640, LC670, LC705, LC Fluro, Cy® 5, and Cy® 5.5 3′ FAM; Acceptor Probe 2 (5′ end): LC® Cyan 500, LC® Fluo, Texas Red, LC® Red 610, LC® Red 640, LC® Red 670, and ROX.

As used herein, the term “hybridization” refers to the binding of two complementary strands of nucleic acid to form a double-stranded molecule (hybrid). The term binding may be used in place of hybridization in the present invention. Hybridization does not require a complete sequence identity between two complementary strands of nucleic acids, some mismatches are acceptable and the nucleic acid strands will still hybridize. The hybridization strength is however determined by the level of sequence identity between the two complementary nucleic acid strands.

A method of making real-time PCR primers for screening a sample is also provided. The method may include i) identifying a target nucleotide sequence of a dermatophyte ESTRP gene, ii) generating a primer pair designed to amplify nucleic acid products containing the target nucleotide sequence, and iii) performing one or more real-time PCRs using the generated primer pair, and optionally assessing (a) a positive control sample that includes the target nucleotide sequence to obtain one or more ranges of one or more Tm values, thereby generating one or more reference Tm ranges, and optionally assessing (b) a negative control sample that does not include the target nucleotide sequence to obtain a range of Ct values, thereby generating a cutoff Ct value, wherein the one or more reference Tm ranges and/or the cutoff Ct value provide for a determination of the presence or absence in a sample of a dermatophyte species.

As used herein, “sequence identity” or “identity” in the context of two nucleic acid sequences makes reference to a specified percentage of residues in the two sequences that are the same when aligned for maximum correspondence over a specified comparison window, as measured by sequence comparison algorithms or by visual inspection.

As used herein, “percentage of sequence identity” means the value determined by comparing two optimally aligned sequences over a comparison window, wherein the portion of the polynucleotide sequence in the comparison window may include additions or deletions (i.e., gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Any suitable methods of alignment of sequences for comparison may be employed. Thus, the determination of percent identity between any two sequences can be accomplished using a mathematical algorithm. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST®, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. Software for performing BLAST® analyses is publicly available through the National Center for Biotechnology Information.

As used herein, a “subject” refers to any animal, such as a mammal like a dog, cat, bird, livestock, and including a human.

“Within” or “between” as used in reference to a number being within a range of numbers, is meant to be inclusive of the values defining the upper and lower limits of the range.

Unless defined otherwise, all technical and scientific terms used herein have the same meaning as commonly understood by one of ordinary skill in the art to which the present disclosure belongs. Any methods and materials similar or equivalent to those described herein can also be used in the practice or testing of embodiments of the present disclosure. All publications mentioned herein are incorporated herein by reference to disclose and describe the methods and/or materials in connection with which the publications are cited.

It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub-combination. All combinations of the embodiments pertaining to the present disclosure are specifically embraced by the present disclosure and are disclosed herein just as if each and every combination was individually and explicitly disclosed.

The present invention relates further to a kit, comprising one or more components suited for carrying out the method of the present invention. For example, the kit may include any other suitable components for storing, transporting and/or carrying out a PCR reaction with the primers and/or probes described herein. The kit may contain a suitable medium, e.g., an aqueous medium. A suitable aqueous medium includes, without limitation, water, a buffer solution, etc. The buffer may be any suitable buffer for storage of primers and/or for carrying out a PCR reaction. The buffer may have any suitable pH, which can be assessed and determined by a skilled person. The suitable medium may be substantially free of enzymes and compounds that degrade nucleic acids, such as nucleases. In some embodiments, the composition is substantially sterile.

In some embodiments, the kit includes, without limitation, a nucleic acid template, primers, one or more polymerases, nucleotides, etc., suitable for performing a PCR reaction to amplify a nucleotide sequence targeted by the primers described herein. The polymerase may be any suitable polymerase, including, without limitation, a thermostable DNA polymerase, such as Taq polymerase, and variants thereof (e.g., commercially available variants of thermostable DNA polymerases). In some embodiments, the kit includes a nucleic acid intercalating dye, such as a fluorescent intercalating dye. The fluorescent intercalating dye may be any suitable DNA intercalating dye for use in real-time PCR, including, without limitation, SYBR® Green, SYTO®9, LCGReen®, Chromofy™ and EvaGreen®.

In some embodiments, the kit includes a probe configured to specifically hybridize to a nucleic acid that contains a nucleotide sequence that is amplified by the primers. The probe may be a fluorescent hybridization probe that changes its fluorescence properties based on whether the probe is hybridized to a target nucleic acid in physical proximity to another probe with a potentially interacting FRET label. Thus, in some embodiments, the probe includes a fluorescent functional group (e.g., fluorescent dye) covalently attached to the probe nucleic acid. The excitation and emission wavelengths of the attached fluorescent dye may be suitably configured to promote a measurable, distance-dependent interaction between the attached dyes of the two probes.

In some embodiments, a control is performed to confirm proper PCR amplification from samples that are subjected to cell lysis and nucleic acid extraction processes. In certain embodiments, the control includes adding an amount of a known nucleic acid to a sample for which the presence or absence of a dermatophyte is to be determined before the sample is processed to lyse cells and extract nucleic acids from the cells, preparing the sample to lyse cells and release cellular nucleic acids, and performing real-time PCR on the sample using primers that amplifies a nucleotide sequence contained in the known nucleic acid.

In some embodiments, the present method further includes generating a report indicating the presence or absence of one or more dermatophytes in a sample subjected to the method steps herein. The report may be provided in any suitable form, including, but not limited to, a report on a physical piece of paper, a report in digital form accessible by a user interface on a computer system (e.g., a web page, or an e-mail), an entry in a database of a patient's medical record, and/or a data file on a non-transient computer readable data-storage medium (e.g., a flash drive, hard drive, compact disc (CD), etc.).

The sample used in the present invention may be any suitable tissue or fluid/liquid sample, preferably comprising a tissue, or a fluid/liquid in which a tissue was present, such as a tissue extract or any environmental sample in which the presence of a dermatophyte is to be detected. The tissue may be obtained from a human or animal subject. In certain embodiments, the sample includes keratinous tissue, such as nail, skin, hair, etc. A nail sample may include portions of toenails or fingernails. Samples from the environment may comprise or be obtained from a scrapings or collection from the ground, for example obtained from an indoor or outdoor floor surface, the sample may comprise or be obtained from a textile, such as clothing, carpet, curtains, chairs, or other items.

In some embodiments, the sample includes 0.01 mg or more, 0.1 mg or more, including 0.5 mg or more, 1 mg or more, 2 mg or more, 5 mg or more, 10 mg or more, 20 mg or more, 50 mg or more, and includes 200 mg or more of relevant sample, such as nail clippings from one or more fingernails and/or toenails, skin scrapings from any site of the body, including the scalp or hair from any site of the body.

In some embodiments, the sample includes nucleic acids, e.g., DNA, at a concentration of 0.0001 ng/μL or more, 0.001 ng/μL or more, 0.01 ng/μL or more, e.g., 0.05 ng/μL or more, 0.1 ng/μL or more, 1.0 ng/μL or more, 5.0 ng/μL or more, 10 ng/μL or more, including 50 ng/μL or more, and includes nucleic acids, e.g., DNA, at a concentration of 1,000 ng/μL or more.

The sample may be prepared to lyse cells and release nucleic acids within cells into a solution using any suitable method, as described below. In some embodiments, the sample contains a suitable buffer for lysing cells, for stabilizing nucleic acids in the sample and/or for carrying out PCRs.

In certain embodiments, the present method includes preparing a sample, e.g., a nail sample, for screening by the method described herein. Preparing the sample may include treating the sample with mechanical, thermal, chemical and/or enzymatic methods of lysing cells and cellular compartments (e.g., plasma membrane, cell wall, nucleus, mitochondria, etc.) in the sample to release nucleic acids, e.g., DNA and/or RNA, into the bulk of the sample. Any suitable method of mechanically lysing cells may be used. In some embodiments, mechanically lysing the cells includes, e.g., homogenizing, grinding, ultrasonicating or freezing the sample. In some embodiments, cells in the sample may be physically lysed by subjecting the sample to a blender, bead or ultrasonic homogenization, grinding by a mortar and pestle, French press, etc.

Any suitable method of chemically lysing cells may be used. In some embodiments, chemical lysis methods include alkaline lysis, detergent lysis (e.g., sodium dodecyl sulfate (SDS)), solvent lysis (e.g., chloroform), etc. In one embodiment, chemically lysing cells involves use of a chaotropic agent, e.g., a chaotropic salt. Non-limiting examples of chaotropic agents include guanidinium isothiocyanate, guanidinium chloride, urea, thiourea, lithium perchlorate, lithium acetate, sodium iodide, phenol and others.

Any suitable method of enzymatically lysing cells may be used. In some embodiments, enzymatic lysis methods include treatment of the sample with protease, lipase, glycoside hydrolases, etc. In some embodiments, cells in the sample may be enzymatically lysed by subjecting the sample to proteinase K, keratinase, trypsin, subtilisin, lyticase, lysozyme, collagenase, cellulase, glucanase, chitinase, pectinase, or amylase, etc.

Any suitable method of thermally lysing cells may be used. In some embodiments, the sample is subjected to a temperature of 50° C. or more, e.g., 60° C. or more, 70° C. or more, 80° C. or more, 90° C. or more, or 95° C. or more, and is subjected to a temperature of 100° C. or less, e.g., 98° C. or less, including 95° C. or less, to lyse the cells in the sample.

The methods of the present disclosure can be in part or in whole computer-implemented, such that method steps (e.g., screening, determining, analyzing, calculating, and/or the like) are automated in whole or in part. Accordingly, the present disclosure provides methods, computer systems, devices, software and the like in connection with computer-implemented methods of detecting a dermatophyte in a sample.

For example, the method steps, including obtaining Ct values and/or Tm values for a real-time PCR, analyzing the Ct values and/or Tm values, comparing Ct values and/or Tm values to cutoff and/or reference values and/or ranges, generating a report, and the like, can be completely or partially performed by a computer program product (software, which may be encompassed by the kit of the invention). Values obtained can be stored electronically, e.g., in a database, and can be subjected to an algorithm executed by a programmed computer. A database may store cutoff and/or reference values and/or ranges that are specific for a dermatophyte and have a database structure that allows retrieval of the cutoff and/or reference values and/or ranges based on an identifying label for the dermatophyte.

The present disclosure provides systems for executing the program described above, which system generally includes: a) a central computing environment; b) an input device, operatively connected to the computing environment, to receive data, wherein the data can include, for example, Ct and/or Tm values or other information obtained from an assay using a sample from a subject, as described above; c) an output device, connected to the computing environment, to provide information to a user (e.g., medical personnel); and d) an algorithm executed by the central computing environment (e.g., a processor), where the algorithm is executed based on the data received by the input device, and wherein the algorithm analyzes the input data to determine the presence or absence of a dermatophyte infection in the sample.

Thus, the present method finds use in diagnosing an infection in a patient, e.g., a human patient, suffering from an infection. The methods of the present disclosure thus may include obtaining a sample, e.g., a nail or other cutaneous sample, determining the presence or absence of dermatophyte in the sample and, if present, the type of dermatophyte, using an assay method as described herein, preferably generating a report that indicates the presence or absence of one or more dermatophytes in the patient sample and, optionally, if present, identifying the likely type of dermatophyte present in the infection, and, optionally, indicating suggested therapy(ies) for treatment of the infection based on the assay results.

Therapeutic Approaches:

Dermatophytes are filamentous fungi in the genera Trichophyton, Microsporum, Epidermophyton and Nannizzia. Dermatophytes metabolize and subsist upon keratin in the skin, hair, and nails. Dermatophyte infections are common worldwide, and dermatophytes are the prevailing causes of fungal infection of the skin, hair, and nails. These infections lead to a variety of clinical manifestations, such as Tinea pedis, Tinea corporis, Tinea cruris, Majocchi's granuloma, Tinea capitis, and Tinea unguium (dermatophyte onychomycosis). Dermatophyte infections of scalp hair (Tinea capitis), beard hair (Tinea barbae), and nails (Tinea unguium) are common and may be diagnosed and/or treated by the present invention.

The major clinical subtypes of dermatophyte infections are: Tinea corporis—Infection of body surfaces other than the feet, groin, face, scalp hair, or beard hair, Tinea pedis—Infection of the foot, Tinea cruris—Infection of the groin, Tinea capitis—Infection of scalp hair, Tinea unguium (dermatophyte onychomycosis)—Infection of the nail.

Tinea corporis, Tinea pedis, Tinea cruris, Tinea faciei, and Tinea manuum infections are typically superficial, involving only the epidermis. Occasionally, dermatophyte infections penetrate the hair follicle and dermis causing a condition called Majocchi's granuloma. Tinea capitis and Tinea barbae are characterized by infection of terminal hairs.

If a cutaneous dermatophyte infection is misdiagnosed and initially treated with antibiotica and/or a topical corticosteroid, the appearance of the infection may be altered, making diagnosis more difficult (i.e., tinea incognito). Patients can develop diminished erythema and scale, loss of a well-defined border, exacerbation of disease, or a deep-seated folliculitis (Majocchi's granuloma). For these reasons, the present invention enables significant improvements in subsequent treatment options, by determining the pathogen to be treated and appropriate therapeutic options.

The methods of the present disclosure can include selecting a therapy, e.g., an antifungal medication, based on the results of the assay. In some embodiments, the methods of the present disclosure can include administering a therapy, e.g., an antifungal medication, based on the results of the assay. Where the methods include selection and/or administration of an antifungal therapy, the therapy is selected according to the dermatophyte detected.

Topical or systemic antifungal drugs with antidermatophyte activity are effective therapies. Most superficial cutaneous dermatophyte infections can be managed with topical therapy with agents such as azoles, allylamines, ciclopirox, and tolnaftate. Oral treatment with agents such as terbinafine, itraconazole, fluconazole, and griseofulvin is used for extensive or refractory cutaneous infections and infections extending into follicles or the dermis (e.g., Majocchi's granuloma) or involving nails.

In some embodiments, the therapy includes administering a pharmaceutical compound or composition. A pharmaceutical compound or drug suitable for treating such an infection may be administered using any suitable method. The pharmaceutical compound may be administered topically or systemically (oral). In some embodiments, a pharmaceutical compound is administered orally and/or topically. An orally administered pharmaceutical compound for treating onychomycosis may include, without limitation, itraconazole, fluconazole, and/or terbinafine. A topically administered pharmaceutical compound for treating onychomycosis may include, without limitation, miconazole, tavaborole, efinaconazole or ciclopirox. The pharmaceutical compound may be administered in any suitable dosage form, e.g., as a tablet, liquid, cream, emulsion, etc. and may be administered in conjunction with any suitable pharmaceutically acceptable carrier.

Anti-mycotic treatments may also include, without limitation, topical agents such as miconazole, terbinafine, clotrimazole, ketoconazole, or tolnaftate, preferably applied twice daily until symptoms resolve, usually within two to six weeks.

Preferred Sequences of the Invention:

| SEQ | | | |
|---|---|---|---|
| ID | | | |
| NO | Sequence 5′-3′ | Name | Gene |
| 1 | ACTCCTCCAAACTACACCCAgCAA | PCR1_for1 | ESTRP |
| 2 | AgCTCTgATCTCACCggTgACgATAg | PCR1_for2 | ESTRP |
| 3 | TgCCCTCAgCAAgAgTTgCAAT | PCR1_rev1 | ESTRP |
| 4 | gCTTggAgggCTggggAgT | PCR1_rev2 | ESTRP |
| 5 | TCACTCTCgTCgTTgCTgCC | PCR2_for1 | ESTRP |
| 6 | CCTAYggCCTCCTgATCgT | PCR2_for2 | ESTRP |
| 7 | ggCAgCCgTggAggAgCA | PCR2_rev1 | ESTRP |
| 8 | TggTggACTTTggTggCT | PCR2_rev2 | ESTRP |
| 9 | gCgCYCgCCRgAggA | PCR1_for3 | ITS |
| 10 | CCggAACCAAgAgATCCgTTg | PCR1_rev3 | ITS |
| 11 | CACATTAACTTggTCgTYATCggC | PCR2_for3 | EF-1-α |
| 12 | gAACTTCTCAATggTACg | PCR2_rev3 | EF-1-α |
| 13 | gCCRCCgTCTCTgCCgTCACTCCTCCAAACTA | ESTRP I | ESTRP |
| | | anchor | |
| 14 | gAgggCACCggCgAgTACCAgTACAgCA | ESTRP II | ESTRP |
| | | anchor | |
| 15 | TCCCTgCCTCCCTCTggCAACCCAA | ESTRP I probe | ESTRP |
| 16 | CAAATTCggCgTCAAgAACgAgggCC | ESTRP II probe | ESTRP |
| 17 | CgATACCACCgCACTTgTAgATCAAgTgACC | EF-1-α anchor | EF-1-α |
| 18 | gTAATgTTATAgTCAgTTTCTgTgTAATTCggT | EF-1-α probe | EF-1-α |
| 19 | ATGAAGGTCACTCTCGTCGTTGCTGCCCTCGCGGCCGCC | Trichophyton | ESTRP |
| | GTCTCTGCCGTCACTCCTCCAAACTACTCCCTGCCTCCCT | verrucosum | |
| | CTGGCAACCCCATCGGCACCCCAGGCCTCCACGAGAAGG | | |
| | TCCCTGTCGACAAGCCCTACGCCATCACCTGGCAGGCAA | | |
| | CCACCGAGAGCCATGTCTCCATCATGCTCCTCCACGGCT | | |
| | GCCCCAAGAACTGCAACCCAGTTCAAACTCTTGCTGAGAA | | |
| | CATCCCGAACACCGGCAGCCTCTCTTGGACTCCTAGTTCT | | |
| | GACCTCACCGGTGACGACTCCTACGGCCTCGTGATCGTC | | |
| | GTCGAGGGCACCGGCCAGTACCAGTACAGCACCAACTTC | | |
| | GGCATCGAGAACCACAGCCCCAAGCCACAGCCACCAAAG | | |
| | TCCACCACGCCAGCCGAGAAGCCTACCTGGACTCCCCAG | | |
| | CCCTCCAAGCCAGTCACCCACATCGTCGAGGCTTCCTCC | | |
| | AGCACTCCCGTCCCCTCCGGCGGCGTCATCACTCTCACC | | |
| | ACCTCCATCTGCCCGCCATCTGCTACCACTTCCACTGTCC | | |
| | CCGGCGTGCCCCAGCCAACTGGCAGCGCGCCAGTCCCC | | |
| | GGCACTCCTCACCCAACCGGCGGCAACCCTGGCCCAGCT | | |
| | CCAGCTCCATCTGGCTCCGGTGCTCCCGTGCCACCACCA | | |
| | GCCTCGACCAACACCCCTCCACCATTCAACAACGGTGCC | | |
| | GGCCGCGTCGGCGCTGGCTTCGGTGCCGCTCTCCTCGTT | | |
| | GTCGCCGCTGCTTTTGCCATGTAA | | |
| re | gi\|291185730\|gb\|ACYE01000206.1\|:17269-18266 | Additional info | |
| 19 | Trichophyton verrucosum Contig00321_1.f1.exp, | | |
| | whole genome shotgun sequence; | | |
| | genome reference number NZ_ACYE00000000, | | |
| | locus_tag TRV_04070 | | |
| 20 | ATGAAGTTCACTCTCGTCGTTGCTGCCTTCGCGGCCACCG | Trichophyton | ESTRP |
| | TCTCTGCCGTCACTCCTCCAAACTACACCCAGCAACCCTC | tonsurans | |
| | CGGCAACCCCATCACTACTCCGGGCCTCGGCGAGCGCGT | | |
| | CCCAGTCGGCCAGGTCTTCACCATCTCCTGGAAGCCAAC | | |
| | CACCCAGAAGCCCGTCTCCATCATGCTCCTCCACGGCTG | | |
| | CCCCCAGAACTGCAACCCAATTGCAACTCTTGCTGAGGG | | |
| | CATCCCCAACTCCGGCTCTCTCCCTTGGACTCCTGAAGCT | | |
| | GATCTCGTCGATGATAACGCCTACGGCCTCCTGATCGTTG | | |
| | TCGAGGGCACCGGCGAGTACCAGTACAGCACCCAATTCG | | |
| | GCGTCAAGAACGACGGCCCCAAGCCACAGCCACCAAAGT | | |
| | CCACCAAGCCAGCCGAGAAGCCTACCTGGGTTCCCCAGC | | |
| | CCTCCAAGCCAGTCACCCACATCATCGACACCGCCTCCA | | |
| | GCACTCCCGTCCCCTCCAGCGGCGTCGTCACCCTCACCA | | |
| | CCTCTGTCTGCCCACCATCTGCCACCACCGTCCCCGGTG | | |
| | TGCCCCAGCCAACCGGCAGCATGCCCATCCCCGGTACTC | | |
| | CTCAGCCAACTGGCGGCAACCCGGGCCCAGCTCCAGCTC | | |
| | CGTCTGGCACCAGTGCTCCCATGCCACCACCAGGCTCGA | | |
| | CCAACACCCCTCCACCATTCAACAACGGTGCCGGCCGCG | | |
| | TCGGCGCTGGTCTCGGTGCCGCTTTCCTCGTTCTCGCCG | | |
| | CTGCTTTTGCCATGTAA | | |
| re | tons_gi\|255681490\|gb\|ACPI01000834.1\|:1846-2836 | Additional info | |
| 20 | Trichophyton tonsurans CBS 112818 cont1.834, | | |
| | whole genome shotgun sequence | | |
| 21 | ATGAAGTTCACTCTCGTCGTTGCTGCCTTCGCGGCCACCG | Trichophyton | ESTRP |
| | TCTCTGCCGTCACTCCTCCAAACTACACCCAGCAACCCTC | equinum | |
| | CGGCAACCCCATCACTACTCCGGGCCTCGGCGAGCGCGT | | |
| | CACAGTCGGCCAGGTCTTCACCATCTCCTGGAAGCCAAC | | |
| | CACCCAGAAGCCCGTCTCCATCATGCTCCTCCACGGCTG | | |
| | CCCCCAGAACTGCAACCCAATTGCAACTCTTGCTGAGGG | | |
| | CATCCCCAACTCCGGCTCTCTCCCTTGGACTCCTGAAGCT | | |
| | GATCTCGTCGATGATAACGCCTACGGCCTCCTGATCGTTG | | |
| | TCGAGGGCACCGGCGAGTACCAGTACAGCACCCAATTCG | | |
| | GCGTCAAGAACGACGGCCCCAAGCCACAGCCACCAAAGT | | |
| | CCACCAAGCCAGCCGAGAAGCCTACCTGGGTTCCCCAGC | | |
| | CCTCCAAGCCAGTCACCCACATCATCGACACCGCCTCCA | | |
| | GCACTCCCGTCCCCTCCAGCGGCGTCGTCACCCTCACCA | | |
| | CCTCCGTCTGCCCACCATCTGCCACCACCGTCCCCGGTG | | |
| | TGCCCCAGCCAACCGGCAGCATGCCCATCCCCGGTACTC | | |
| | CTCAGCCAACCGGCAGCGTGCCCATCCCTGGCACTCAGC | | |
| | CAACTGGCGGCAACCCGGGCCCAGCTCCAGCTCCGTCTG | | |
| | GCACCAGTGCTCCCATGCCACCACCAGGCTCGACCAACA | | |
| | CCCCTCCACCATTCAACAACGGTGCCGGCCGCGTCGGCG | | |
| | CTGGTCTCGGTGCCGCTTTCCTCGCTCTCGCCGCTGCTTT | | |
| | TGCCATGTAA | | |
| re | gi\|209488040\|gb\|ABWI01001154.1\|:2868-3894_(reversed) | Additional info | |
| 21 | Trichophyton equinum CBS 127.97 cont1.1154, | | |
| | whole genome shotgun sequence | | |
| 22 | ATGAAGTTCACTCTCGTCGTTGCTGCCTTCGCGGCCACCG | Trichophyton | ESTRP |
| | TCTCTGCCGTCACTCCTCCAAACTACACCCAGCAACCCTC | interdigitale | |
| | CGGCAACCCCATCACTGCTCCGGGCCTCGGCGAGCGCG | | |
| | TCCCAGTCGGCCAGGTCTTCACCATCTCCTGGCAGCCAA | | |
| | CCACCCAGAAGCCCGTCTCCCTCATGCTCCTCCACGGCT | | |
| | GCCCCCTGAACTGCAACCCAATTGCAACTCTTGCTGAGG | | |
| | GCATCCCAAACTCCGGCTCTCTCCCTTGGACTCCTGAAGC | | |
| | TGGTCTCGTCGATGATGACGCCTACGGCCTCCTGATCGTT | | |
| | GTCGAGGGCACCGGCGAGTACCAGTACAGCACCAAATTC | | |
| | GGCGTCAAGAACGAGGGCCCCAAGCCACAGCCACCAAAG | | |
| | TCCACCAAGCCAGCCGAGAAGCCTACCTGGGTTCCCCAG | | |
| | CCCTCCAAGCCAGTCACCCACATCATCGACACCGCCTCC | | |
| | AGCACTCCCGTCCCCTCCAGCGGCGTCGTCACCCTCACC | | |
| | ACCTCCGTCTGCCCACCATCTGCCACCACAGTCCCCGGT | | |
| | GTGCCCCAGCCAACCGGCAGCATGCCCATCCCCGGTACT | | |
| | CCTCAGCCAACCGGCAGCGTGCCCATCCCTGGCACTCCT | | |
| | CAGCCAACTGGCGGCAACCCGGGCCCAGCTCCAGCTCCA | | |
| | TCTGGCACCACTGCTCCCATGCCACCACCAGGCTCGACC | | |
| | AACACCCCTCCACCATTCAACAACGGTGCCGGCCGCGTC | | |
| | GGCGCTGGTCTCGGTGCCGCTTTCCTCGTTCTCGCCGCT | | |
| | GCTTTTGCCATGTAA | | |
| re | gi\|604714071\|gb\|AOKY01000705.1\|:10-861 | Additional info | |
| 22 | Trichophyton interdigitale Cont1.705, | | |
| | whole genome shotgun sequence | | |
| 23 | ATGAAGGTCACTCTCGTCGTTGCTGCCCTCGCGGCCGCC | Trichophyton | ESTRP |
| | GTCTCTGCCGTCACTCCTCCAAACTACTCCCAGCCTCCCT | benhamiae | |
| | CCGGCAACCCCATCGGCACCCCAGGCCTCCACGAGAAG | | |
| | GTCCCTGTCGGCCAGCCCTACACCATCACCTGGCAGGCA | | |
| | ACCACCGAGAGCCATGTCTCCATCATGCTCCTCCACGGCT | | |
| | GCCCCAAGAACTGCAACCCAGTTCAAACTCTTGCTGAGAA | | |
| | CATCCCGAACTCCGGCAGCCTCTCTTGGACTCCCAAGTC | | |
| | CGACCTCACCGGTGACGACTCCTACGGCCTCATGATCGT | | |
| | CGTCGAGGGCACCGGCCAGTACCAGTACAGCACCAACTT | | |
| | CGGCATCGAGAACCACAGCCCCAAGCCACAGCCACCAAA | | |
| | GTCCACCACGCCAGTCGAGAAGCCTACCTGGACTCCCCA | | |
| | GCCCTCCAAGCCAGTCACCCACATCGTCGAGACTTCCTC | | |
| | CAGCACTCCCGTCCCCTCCGGCGGCGTCATCACTCTCAC | | |
| | CACCTCCATCTGCCCGCCATCTGCTACCACTTCCACTGTC | | |
| | CCCGGCGTGCCCCAGCCAACTGGCAGCGCGCCAGTACC | | |
| | CGGCACTCCTCACCCAACCGGCGGCAACCCTGGACCAGC | | |
| | TCCAGCTCCATCTGGCTCCGGTGCTCCCGTGCCACCACC | | |
| | AGCCTCGACCAACACCCCTCCACCATTCAACAACGGTGC | | |
| | CGGCCGCGTIGGCGCTGGTTTCGGIGCCGCTCTCCTCGT | | |
| | TGTCGCCGCTGCTTTTGCCATGTAA | | |
| re | benh_gi\|291180644\|gb\|ABSU01000001.1\|: | Additional info | |
| 23 | 1406842-1408608_(reversed) Trichophyton | | |
| | benhamiae white type Contig01293.0, | | |
| | whole genome shotgun sequence | | |
| 24 | ATGAAGCTCACTCTCGTCGTTGCTGCCCTCGCGGCCGCC | Trichophyton | ESTRP |
| | GTCTCTGCCGTCACTCCTCCAAACTACAGCCAGTCTCCCT | rubrum | |
| | CCGGCAACCCCATCGCTTCCCCGGGCCTCCACGAGCGC | | |
| | GTCCCTGTCAGCAAGCCCTACACCATCACCTGGCAGGCA | | |
| | ACCACCTCGAGCCACGTCTCCATCATGCTCCTCCACGGCT | | |
| | GCCCCAAGAACTGCGAACCAGTTGCAACGCTTGCGGAGA | | |
| | ACATCCCCAACTCCGGCCACCACTCCTGGACTCCTAGCTC | | |
| | TGATCTCACCGGTGACGATAGCTACGGCCTCATGATTGTC | | |
| | GTCGAGGGCACCGGCCAGTACCAGTACAGCACCAACTTC | | |
| | GGCATCGAGAACCACAGCCCCAAGCCACAGCCACCAAAG | | |
| | TCCACCACGCCAGCCGAGAAGCCTACCTGGACTCCCCAG | | |
| | CCCTCCAAGCCAGTCACCCATATCATCGAGACCTCCAGCA | | |
| | CTCCCGTCCCCTCCGGCGGCGTCATCACTCTCACCACCT | | |
| | CCATCTGCCCTCCATCCGGCACCGCCGTCCCCGGTGTGC | | |
| | CCCATCCAACCGGCAGCGTGCCTGTCCCCGGCACTCCTC | | |
| | ACCCAACCGGCGGCAACCCAGGCCCAGCTCCAGCTCCAT | | |
| | CTGGCTCTGGTGCTCCCATGCCACCACCAGCCTCGACCA | | |
| | ACACCCCTCCACCATTCAACAACGGTGCCGGCCGCGTCG | | |
| | GCGCTGGTCTCGGTGCCGCTCTCCTCGTTGTCGCCGCTG | | |
| | CTTTTGCCATGTAA | | |
| re | gi\|604718901\|gb\|AOKX01000151.1\|:18964-19933 | Additional info | |
| 24 | Trichophyton rubrum CBS 202.88 cont2.151, | | |
| | whole genome shotgun sequence; | | |
| | gi\|604717478\|gb\|AOLB01000143.1\|:36264-37233 | | |
| | Trichophyton rubrum Cont1.143, whole genome | | |
| | shotgun sequence; | | |
| | gi\|604717381\|gb\|AOLC01000137.1\|:24866-25835 | | |
| | Trichophyton rubrum MR1459 cont1.137, whole | | |
| | genome shotgun sequence; | | |
| | gi\|604712815\|gb\|AOKU01000156.1\|:37794-38763 | | |
| | Trichophyton rubrum CBS 288.86 cont1.156, | | |
| | whole genome shotgun sequence | | |
| 25 | ATGAAGCTCACTCTCGTCGTTGCTGCCCTCGCGGCCGCC | Trichophyton | ESTRP |
| | GTCTCTGCCGTCACTCCTCCAAACTACAGCCAGTCTCCCT | soudanense | |
| | CCGGCAACCCCATCGCTTCCCCGGGCCTCCACGAGCGC | | |
| | GTCCCTGTCAGCAAGCCCTACACCATCACCTGGCAGGCA | | |
| | ACCACCTCGAGCCACGTCTCCATCATGCTCCTCCACGGCT | | |
| | GCCCCAAGAACTGCGAACCAGTTGCAACGCTTGCGGAGA | | |
| | ACATCCCCAACTCCGGCCACCACTCCTGGACTCCTAGCTC | | |
| | TGATCTCACCGGTGACGATAGCTACGGCCTCATGATTGTC | | |
| | GTCGAGGGCACCGGCCAGTACCAGTACAGCACCAACTTC | | |
| | GGCATCGAGAACCACAGCCCCCAGCCACAGCCACCAAAG | | |
| | TCCACCACGCCAGCCGAGAAGCCTACCTGGACTCCCCAG | | |
| | CCCTCCAAGCCAGTCACCCATATCATCGAGACCTCCAGCA | | |
| | CTCCCGTCCCCTCCGGCGGCGTCATCACTCTCACCACCT | | |
| | CCATCTGCCCTCCATCCGGCACCGCCGTCCCCGGTGTGC | | |
| | CCCATCCAACCGGCAGCGTGCCTGTCCCCGGCACTCCTC | | |
| | ACCCAACCGGCGGCAACCCAGGCCCAGCTCCAGCTCCAT | | |
| | CTGGCTCTGGTGCTCCCATGCCACCACCAGCCTCGACCA | | |
| | ACACCCCTCCACCATTCAACAACGGTGCCGGCCGCGTCG | | |
| | GCGCTGGTCTCGGTGCCGCTCTCCTCGTTGTCGCCGCTG | | |
| | CTTTTGCCATGTAA | | |
| re | gi\|604714209\|gb\|AOKW01000116.1\|:3444-4413 | Additional info | |
| 25 | Trichophyton soudanense CBS 452.61 cont1.116, | | |
| | whole genome shotgun sequence | | |
| 26 | ATGAAGCTCACTCTCGTCGTTGCTGCCCTCGCGGCCGCC | Trichophyton | ESTRP |
| | GTCTCTGCCGTCACTCCTCCAAACTACAGCCAGTCTCCCT | violaceum | |
| | CCGGCAACCCCATCGCTTCCCCGGGCCTCCACGAGCGC | | |
| | GTCCCTGTCAGCAAGCCCTACACCATCACCTGGCAGGCA | | |
| | ACCACCTCGAGCCACGTCTCCATCATGCTCCTCCACGGCT | | |
| | GCCCCAAGAACTGCGAACCAGTTGCAACGCTTGCGGAGA | | |
| | ACATCCCCAACTCCGGCCACCACTCCTGGACTCCTAGCTC | | |
| | TGATCTCACCGGTGACGATAGCTACGGCCTCATGATTGTC | | |
| | GTCGAGGGCACCGGCCAGTACCAGTACAGCACCAATTTC | | |
| | GGCATCGAGAACCACAGCCCCAAGCCACAGCCACCAAAG | | |
| | TCCACCACGCCAGCCGAGAAGCCTACCTGGACTCCCCAG | | |
| | CCCTCCAAGCCAGTCACCCATATCATCGAGACCTCCAGCA | | |
| | CTCCCGTCCCCTCCGGCGGCGTCATCACTCTCACCACCT | | |
| | CCATCTGCCCTCCATCCGGCACCGCCGTCCCCGGTGTGC | | |
| | CCCATCCAACCGGCAGCGTGCCTGTCCCCGGCACTCCTC | | |
| | ACCCAACCGGCGGCAACCCAGGCCCAGCTCCAGCTCCAT | | |
| | CTGGCTCTGGTGCTCCCATGCCACCACCAGCCTCGACCA | | |
| | ACACCCCTCCACCATTCAACAACGGTGCCGGCCGCGTCG | | |
| | GCGCTGGTCTCGGTGCCGCTCTCCTCGTTGTCGCCGCTG | | |
| | CTTTTGCCATGTAA | | |
| re | gi\|1030006227\|gb\|LHPN01000007.1\|:725095-726064 | Additional info | |
| 26 | Trichophyton violaceum Scaffold7, whole genome | | |
| | shotgun sequence | | |
| 27 | ATCATTAACGCGCAGGCCGGAGGCTGGCCCCCCACGATA | | ITS |
| | GGGACCGACGTTCCATCAGGGGTGAGCAGACGTGCGCC | | |
| | GGCCGTACGCCCCCATTCTTGTCTACCTCACCCGGTTGC | | |
| | CTCGGCGGGCCGCGCTCCCCCTGCCAGGGAGAGCCGTC | | |
| | CGGCGGGCCCCTTCTGGGAGCCTCGAGCCGGACCGCGC | | |
| | CCGCCGGAGGACAGACACCAAGAAAAAATTCTCTGAAGA | | |
| | GCTGTCAGTCTGAGCGTTTAGCAAGCACAATCAGTTAAAA | | |
| | CTTTCAACAACGGATCTCTTGGTTCCGGCATCGATGAAGA | | |
| | ACGCAGCGAAATGCGATAAGTAATGTGAATTGCAGAATTC | | |
| | CGTGAATCATCGAATCTTTGAACGCACATTGCGCCCTCTG | | |
| | GCATTCCGGGGGGCATGCCTGTTCGAGCGTCATTTCAAC | | |
| | CCCTCAAGCCCGGCTTGTGTGATGGACGACCGTCCGGCC | | |
| | CCTCCCTTCGGGGGCGGGACGCGCCCGAAAAGCAGTGG | | |
| | CCAGGCCGCGATTCCGGCTTCCTAGGCGAATGGGCAGCC | | |
| | AATTCAGCGCCCTCAGGACCGGCCGCCCTGGCCCCAATC | | |
| | TTTATATATATATATATCTTTTCAGGTTGACC | | |
| re | NR_131330.1 Trichophyton rubrum CBS 392.58 | Additional info | |
| 27 | ITS region; from TYPE material | | |
| 28 | CACATTAACTTGGTCGTTATCGGCCACGTCGATTCCGGCA | | EF-1-α |
| | AATCCACCACTACTGGTAAGCCAGCCACCAGATACCTCTA | | |
| | GCCAGGCACCGAATTACACAGAAACTGACTATAACATTAC | | |
| | AGGTCACTTGATCTACAAGTGCGGTGGTATCGACCAGCGT | | |
| | ACCATTGAGAAGTTCGAGAAGGTAAATAACCCCCCTTTTTT | | |
| | GACCCTGCTGTCTCCGTTCTGTTGCACAATTTTCCCCCTT | | |
| | CATCCCACTACAGGTGAAATTTTGGTGCTGCTGGTGGGAT | | |
| | GTGGCTTGGCACTCGCTTGGGCAGCAAAATCCACACCCC | | |
| | ACCAACATCAAACATGCAGCCATCGCTCCAGGCAACGATC | | |
| | GAGCCTCATGTCATGTTTGGGATTTGCTTTTTTCTAAGGAT | | |
| | CGATGCTAACAAGGTACCTGTAGGAAGCCGAAGAGTTGG | | |
| | GCAAGAAGTCCTTCAAGTACGCTTGGGTTCTTGACAAGCT | | |
| | CAAGGCCGAGCGTGAGCGTGGTATCACCATCGATATCGC | | |
| | CCTCTGGAAGTTCGAGACCCCCAAGTACAATGTCACCGTC | | |
| | ATTGGTATGTTTCTTTGCCTTGTTCCCTCATGTGGTTGTAC | | |
| | CATATCTAACGAGAGTAGACGCCCCCGGTCACCGTGACTT | | |
| | CATCAAGAACATGATCACTGGTACCTCCCAGGCTGACTGC | | |
| | GCTATTCTCATCATTGCTGCCGGTACTGGTGAGTTCGAGG | | |
| | CTGGTATCTCCAAGGAT | | |
| re | KM678081.1 Trichophyton rubrum strain CBS 304.60 | Additional info | |
| 28 | translation elongation factor 1-alpha (tef-1-alpha) | | |
| | gene, partial cds | | |
| 29 | TCACTCTCGTCGCTGCTGCC | Novel target 1 | ESTRP |
| | | PCR2 for | |
| | | primer for1 | |
| 30 | TCACTCTCGTCGTCGCTGCC | Novel target 2 | ESTRP |
| | | PCR2 for | |
| | | primer for1 | |
| 31 | GCCGTCGCCTCTGCCGTCACTCCTCCAAACTA | Novel target 3 | ESTRP |
| | | PCR2 ESTRP-I | |
| | | for anchor | |
| | | probe | |
| 32 | ACCCAGCAACCCTCCGGCAACCCCA | Novel target 4 | ESTRP |
| | | PCR2 ESTRP-I | |
| | | for species | |
| | | probe | |
| 33 | TCCCAGCAACCCTCCGGCAACCCCA | Novel target 5 | ESTRP |
| | | PCR2 ESTRP-I | |
| | | for species | |
| | | probe | |
| 34 | GCCCAGCAACCCTCCGGCAACCCCA | Novel target 6 | ESTRP |
| | | PCR2 ESTRP-I | |
| | | for species | |
| | | probe | |
| 35 | AGCCAGGATCCCTCCGGCAACCCCT | Novel target 7 | ESTRP |
| | | PCR2 ESTRP-I | |
| | | for species | |
| | | probe | |
| 36 | TCCCAGCCTCCCTCCGGCAACCCCA | Novel target 8 | ESTRP |
| | | PCR2 ESTRP-I | |
| | | for species | |
| | | probe | |
| 37 | TCCCTGCCTCCCTCTGGCAACCCCA | Novel target 9 | ESTRP |
| | | PCR2 ESTRP-I | |
| | | for species | |
| | | probe | |
| 38 | AGCCAGTCTCCCTCCGGCAACCCCA | Novel target 10 | ESTRP |
| | | PCR2 ESTRP-I | |
| | | for species | |
| | | probe | |
| 39 | TCCCAGTCGCCATCCGGCAACCCCA | Novel target 11 | ESTRP |
| | | PCR2 ESTRP-I | |
| | | for species | |
| | | probe | |
| 40 | TCCAACTCTCCATCCGGCAACCCCA | Novel target 12 | ESTRP |
| | | PCR2 ESTRP-I | |
| | | for species | |
| | | probe | |
| 41 | TCCAACTCGCCCTCCGGCAACCCCA | Novel target 13 | ESTRP |
| | | PCR2 ESTRP-I | |
| | | for species | |
| | | probe | |
| 42 | CCTACGGCCTCGTGATCGT | Novel target 14 | ESTRP |
| | | PCR2 for | |
| | | primer for2 | |
| 43 | CCTACGGCCTCATGATCGT | Novel target 15 | ESTRP |
| | | PCR2 for | |
| | | primer for2 | |
| 44 | GCTACGGCCTCATGATTGT | Novel target 16 | ESTRP |
| | | PCR2 for | |
| | | primer for2 | |
| 45 | GAGGGCACCGGCCAGTACCAGTACAGCA | Novel target 17 | ESTRP |
| | | PCR2 ESTRP- | |
| | | II for anchor | |
| | | probe | |
| 46 | CCAATTCGGCGTCAAGAACGAGGGCC | Novel target 18 | ESTRP |
| | | PCR2 ESTRP- | |
| | | II for species | |
| | | probe | |
| 47 | CAACTTCGGCATCGAGAACAACAGCC | Novel target 19 | ESTRP |
| | | PCR2 ESTRP- | |
| | | II for species | |
| | | probe | |
| 48 | CAACTTCGGCGTCGAGAACCACAGCC | Novel target 20 | ESTRP |
| | | PCR2 ESTRP- | |
| | | II for species | |
| | | probe | |
| 49 | CAATTTCGGCATCGAGAACCACAGCC | Novel target 21 | ESTRP |
| | | PCR2 ESTRP- | |
| | | II for species | |
| | | probe | |

According to the present invention, a further aspect relates to the oligonucleotides comprising or consisting of SEQ ID NO 1-8, preferably in the form of a primer; or SEQ ID NO 13-16, preferably in the form of a probe, or to oligonucleotides comprising or consisting of SEQ ID NO 1-18.

The invention therefore relates to
a) Oligonucleotides consisting or comprising of a sequence according to SEQ ID NO 1-18,
b) Oligonucleotides of a) that comprise a 0 to 5 nucleotide addition or deletion at the 5′ and/or 3′ end of a sequence according to SEQ ID NO 1-18,
c) Oligonucleotides of more than 70%, 75%, 80%, 85%, 90% or preferably more than 95% sequence identity to a) or b), and/or
d) Oligonucleotides of a complementary sequence of a) to c).

The oligonucleotide sequence of primers or probes may be derived from either strand of the duplex target DNA. The primers or probes may consist of the bases A, G, C, or T or analogs thereof and they may be degenerated at one or more chosen nucleotide position(s) to ensure DNA amplification for potentially all strains of a target fungal species. Degenerated primers are primers which have a number of possibilities at mismatch positions in the sequence in order to allow annealing to complementary sequences and amplification of a variety of related sequences. Degeneracies reduce the specificity of the primer(s), meaning mismatch opportunities are greater, and background noise increases; also, increased degeneracy means concentration of the individual primers decreases. Degenerated primers should be carefully designed in order to avoid affecting the sensitivity and/or specificity of the assay. Inosine is a modified base that can bind with any of the regular base (A, T, C or G). Inosine is used in order to minimize the number of degeneracies in an oligonucleotide.

As used herein the term “a 0 to 5 nucleotide addition or deletion at the 5′ and/or 3′ end of a sequence means that the oligonucleotide or nucleic acid may have a) 0, 1, 2, 3, 4 or 5 additional nucleotides at its 5′ end and 0, 1, 2, 3, 4 or 5 nucleotides deleted at its 3′ end or b) 0, 1, 2, 3, 4 or 5 additional nucleotide at its 3′ end and 0, 1, 2, 3, 4 or 5 nucleotides deleted at its 5′ end, c) 0, 1, 2, 3, 4 or 5 additional nucleotide at its 5′ end and 0, 1, 2, 3, 4 or 5 additional nucleotides at its 3′ end or d) 0, 1, 2, 3, 4 or 5 nucleotide deleted at its 5′ end and 0, 1, 2, 3, 4 or 5 nucleotides deleted at its 3′ end. Minor changes in the length of the primers and probes described herein may be carried out by a skilled person without undue effort.

In one embodiment, the method of the present invention is characterized in that the one or more primer or probe comprises a nucleotide sequence with more 70%, 75%, 80%, 85%, 90% or preferably more than 95% sequence identity, to the sequences provided herein, for example, 70, 75, 80, 81, 82, 83, 84, 85, 86, 87, 88, 89, 90, 91, 92, 93, 94, 95, 96, 97, 98, 99% sequence identity. The sequence variant with between 70% and 99% sequence identity is preferably functionally analogous, i.e. sequences that although exhibiting differences in DNA sequence, so that for example the same, fewer or more mismatches may occur between primer/probe and target, exhibit a similar Tm and/or binding specificities for the various pathogens to be detected, so that the function of the primer/probe in the context of the present invention is maintained. Functionally analogous sequences can be tested by one skilled in the art without inventive effort in light of the information provided within the application, for example by testing Tm or hybridization properties in the context of the PCR or other amplification methods described.

### FIGURES

The invention is further described by the following figures. These are not intended to limit the scope of the invention, but represent preferred embodiments of aspects of the invention provided for greater illustration.

FIG. 1: Workflow and evaluation of a preferred embodiment of assessing genomic DNA for the presence of a pathogenic dermatophyte via amplification of an ESTRP gene. Tm=melting temperature. Tm1 caused by melting of the PCR product in the ESTRP gene, Tm2 caused by melting of the PCR product in the ITS region.

FIG. 2: Sequence alignment of the binding sites of primers and probes of dermatophyte species, which are discriminated within the ESTRP gene in PCR2, targeting two different target regions of the ESTRP gene (A and B, respectively), using the oligonucleotide sequences SEQ ID NO 5-8 as primers and SEQ ID NO 13-16 as probes. All sequences are given in 5′-3′-direction. Variation in the sequence across the target regions indicated is shown by stars and dots.

FIG. 3: Melting temperatures (Tm) obtained in multiplex PCR 1. ESTRP (upper) and ITS (lower) results are shown. A unique Tm combination is obtained for T. rubrum with Tm1 between 79.9° C. and 80.4° C. and Tm2 between 87.0° C. and 87.5. 1=T. tonsurans, 2=T. equinum, 3=T. interdigitale, 4=T. mentagrophytes, 5=T. quinckeanum, 6=T. benhamiae, 7=T. erinacei, 8=T. verrucosum, 9=T. rubrum, 10=T. violaceum, 11=E. floccosum, 12=Nannizzia spp. 13=M. canis, 14=M. audouinii, 15=Paraphyton spp., 16=Lophophyton gallinae, 17=Arthroderma spp.

FIG. 4: Matrix representation of DNA sequence identities of the ESTRP coding regions in the various organisms (ESTRP gene sequences previously available from NCBI database).

FIG. 5: Matrix representation of DNA sequence identities of the ESTRP coding regions in the various organisms (ESTRP gene sequencing was carried out for the ESTRP gene of each of said dermatophyte, listed as T. tonsurans 1, T. equinum 2, T. interdigitale 3, T. mentagrophytes 4, T. quinckeanum 5, T. schoenleinii 6, T. simii 7, T. erinacei 8, T. concentricum 9, T. benhamiae 10, T. verrucosum 11, T. rubrum 12, T. soudanense 13, T. violaceum 14, E. floccosum 15, N. persicolor 16, N. gypsea 17, N. fulva 18, N. incurvata 19, M. canis 20, M. ferrugineum 21).

FIG. 6: Matrix representation of the number of DNA sequence differences of the ESTRP coding regions in the various organisms (ESTRP gene sequences previously available from NCBI database).

FIG. 7: Matrix representation of the number of DNA sequence differences of the ESTRP coding regions in the various organisms (ESTRP gene sequencing was carried out for the ESTRP gene of each of said dermatophyte, listed as T. tonsurans 1, T. equinum 2, T. interdigitale 3, T. mentagrophytes 4, T. quinckeanum 5, T. schoenleinii 6, T. simii 7, T. erinacei 8, T. concentricum 9, T. benhamiae 10, T. verrucosum 11, T. rubrum 12, T. soudanense 13, T. violaceum 14, E. floccosum 15, N. persicolor 16, N. gypsea 17, N. fulva 18, N. incurvata 19, M. canis 20, M. ferrugineum 21).

FIG. 8: Sequence alignment of the ESTRP genes of T. interdigitale, T. tonsurans, T. equinum, T. soudanense, T. violaceum, T. rubrum, T. benhamiae and T. verrucosum (SEQ ID NO 19-26).

### EXAMPLES

The invention is further described by the following examples. These are not intended to limit the scope of the invention, but represent preferred embodiments of aspects of the invention provided for greater illustration.

The present invention has employed the genome sequence of a T. tonsurans strain published by the Broad Institute (Cambridge, USA) and a T. equinum strain, in order to screen for alternative target genes suitable as diagnostic markers for differentiating between phylogenetically closely related dermatophyte species. The focus of the marker search was in light of employing quantitative real-time PCR (qRT-PCR), preferably in combination with a melting curve analysis.

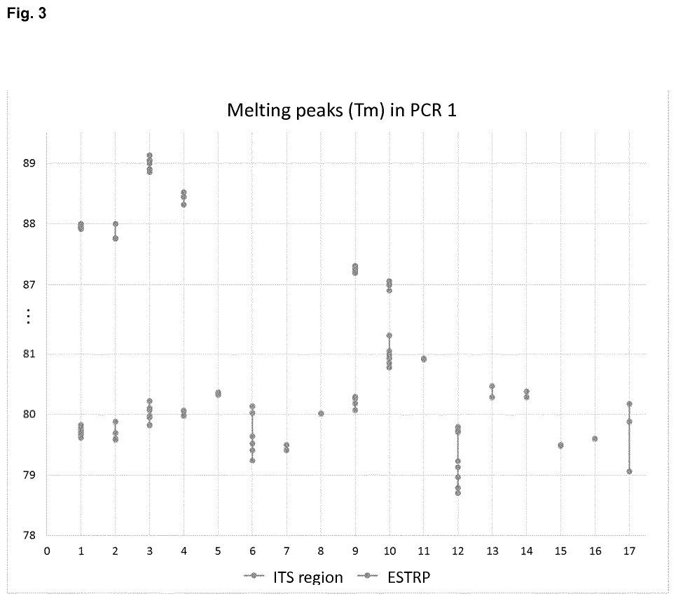

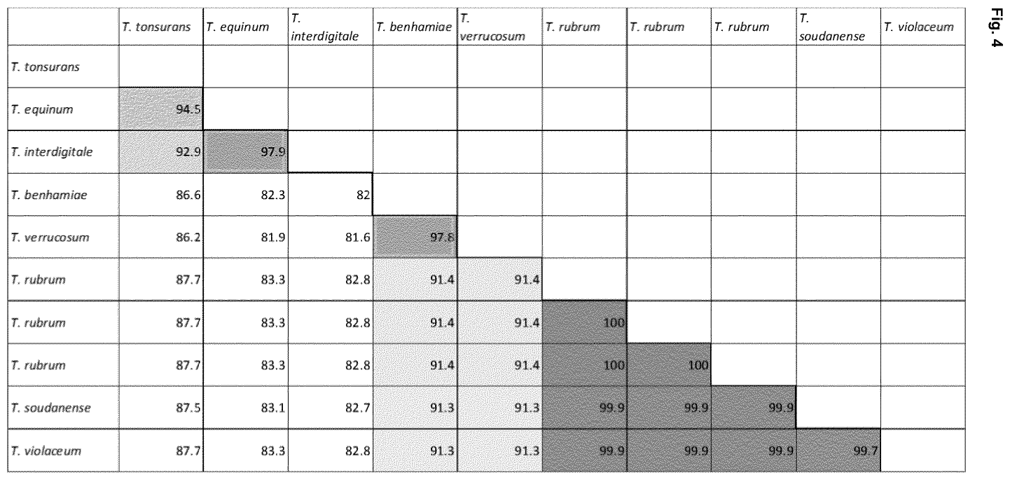

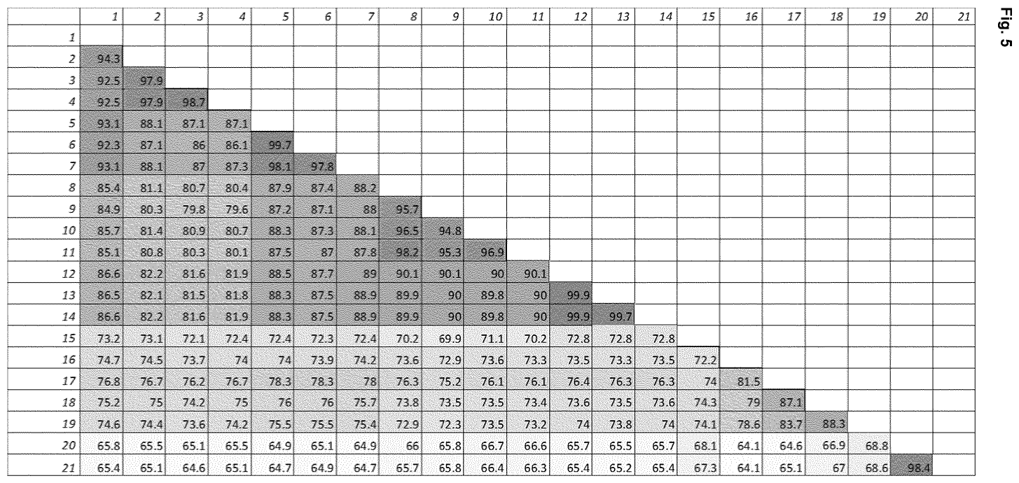

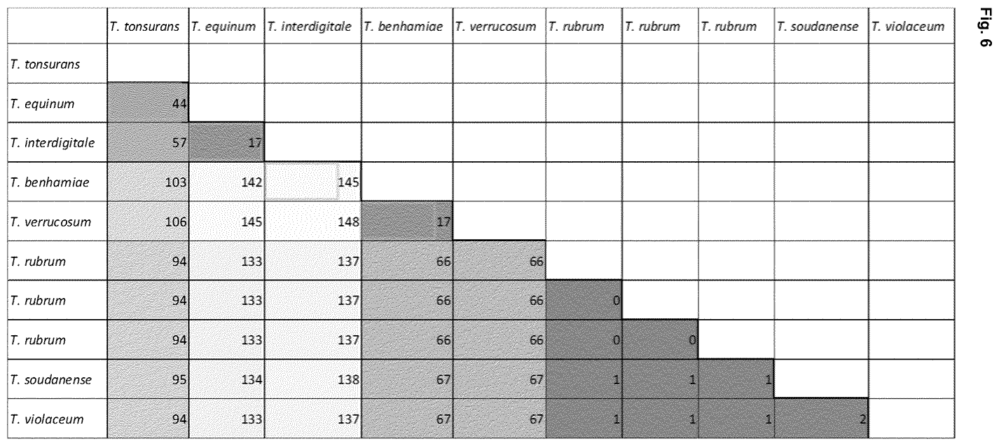

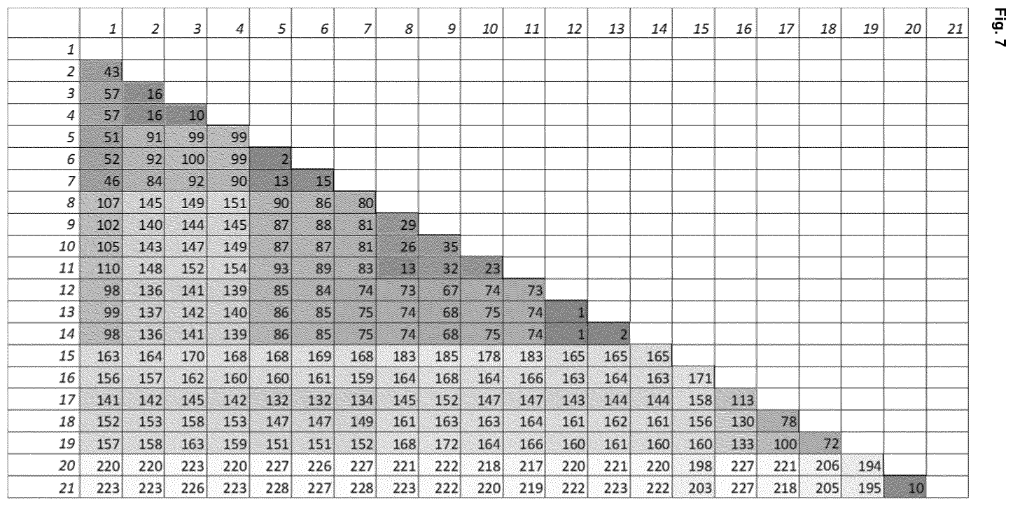

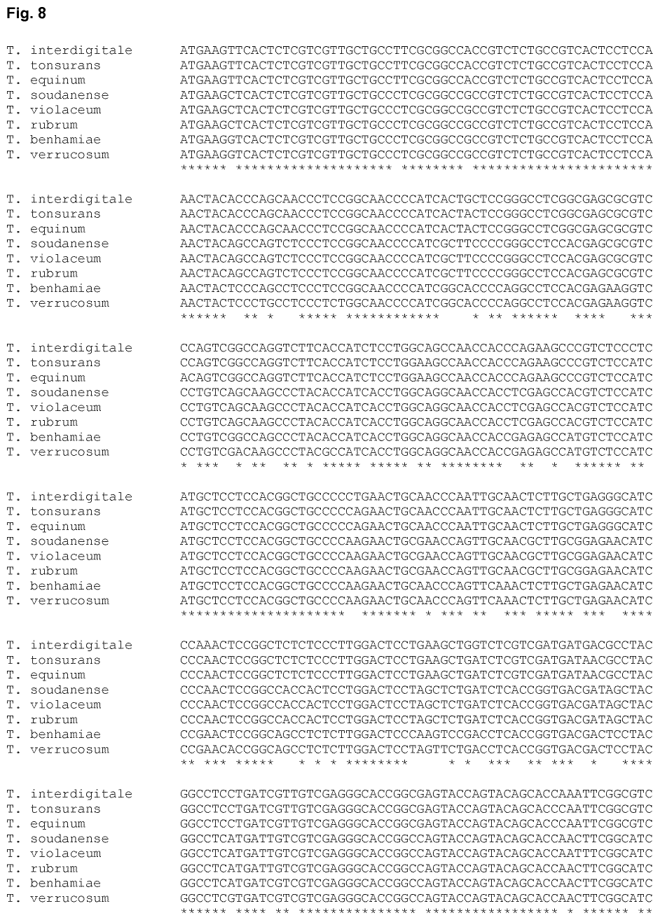

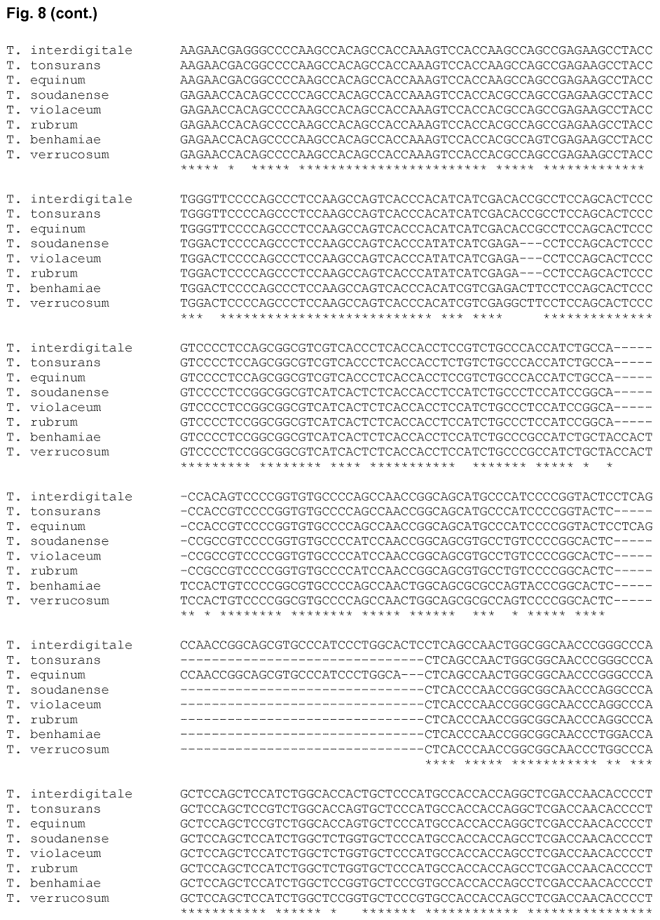

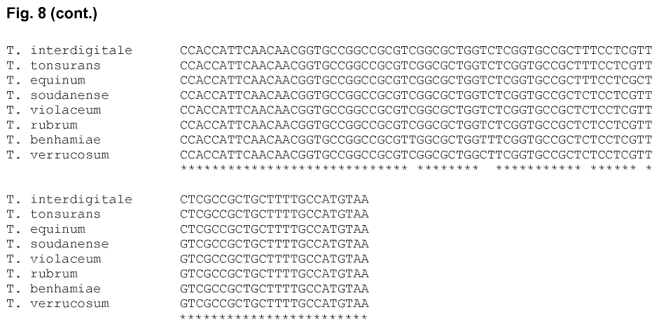

In the prototype experiments, the inventors were able to show that the dermatophyte-specific detection works with EvaGreen. Different dermatophyte species could be detected, while selected non-dermatophyte species (Aspergillus candidus, Aspergillus versicolor, Candida dubliniensis, Chrysosporium articulatum, Fusarium orxysporum, Fusarium solani, Scopulariopsis brevicaulis) did not produce a signal or merely a nonspecific signal at 77° C. Id. The inventors have also shown that it is possible to differentiate the common species T. rubrum from other species in the first reaction (PCR1; FIG. 3).

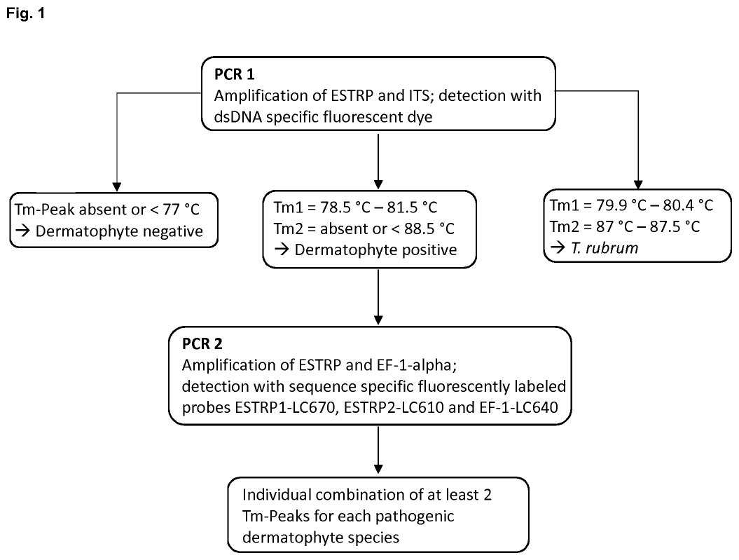

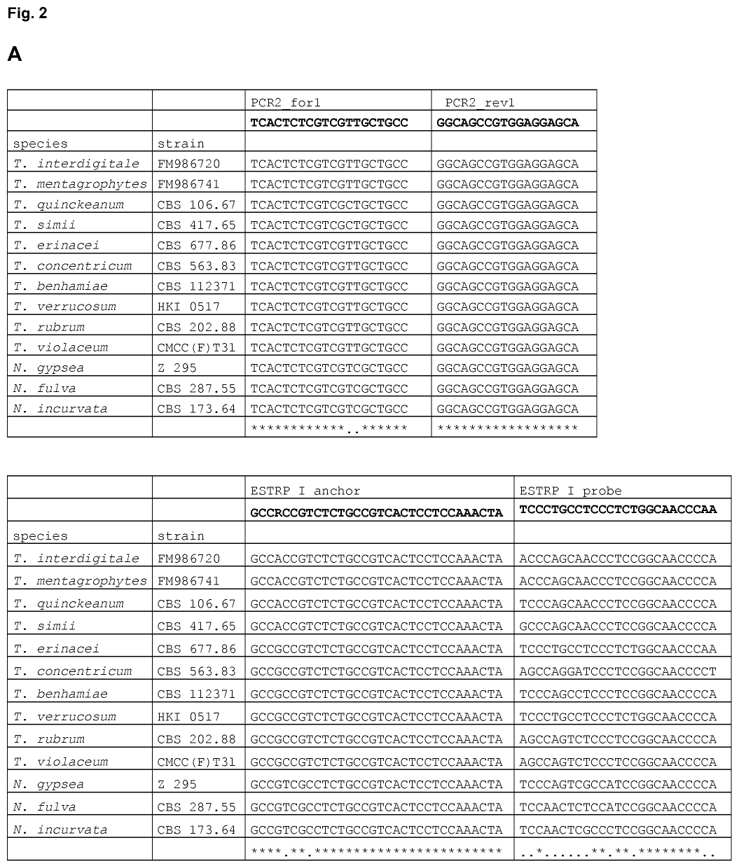

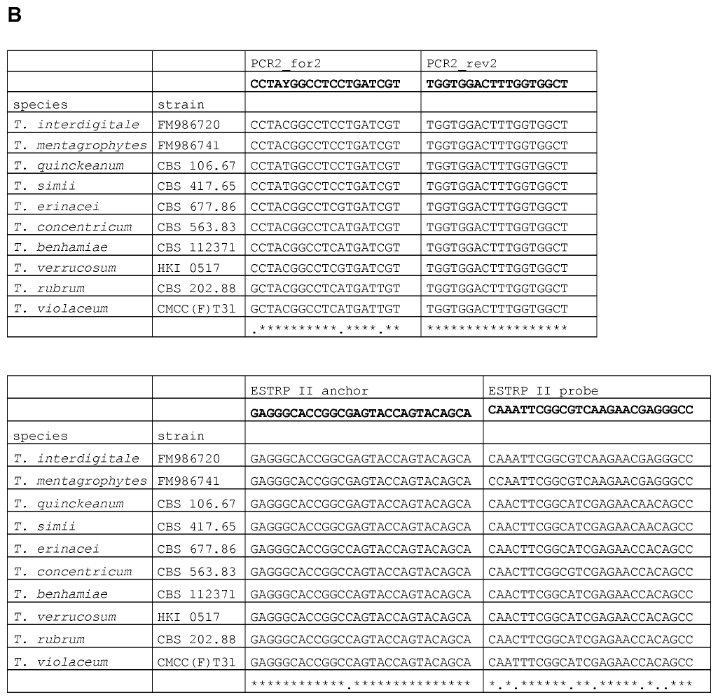

The ESTRP gene includes at least two variable regions responsible for the development of species-group specific primers for a first PCR (PCR 1; EvaGreen (or other dsDNA binding dye) supported identification of T. rubrum, T. violaceum and differentiation between T. interdigitale and T. mentagrophytes (FIG. 1 and FIG. 3). Species-specific FRET probes are then suitable for probe-assisted differentiation of the remaining dermatophytes in a second PCR (PCR 2).

For the universal detection of dermatophytes, as well as the differentiation of T. rubrum and T. violaceum, a section of the ITS region is, in some embodiments, additionally used in PCR 1.

In PCR 2, in preferred embodiments, the differentiation of species using a gene region of the translation elongation factor 1-alpha is employed.

By constructing the method, in preferred embodiments, as a multiplex PCR (preferably amplification of the ITS region and ESTRP in PCR 1, and EF-1-alpha and ESTRP in PCR 2) with the simultaneous use of three probes bound with three different fluorescent dyes (eg. LC610, LC640 and LC670), all relevant species can be analysed with only two PCR reactions (FIG. 1).

The universal dermatophyte detection of the first reaction therefore preferably takes place on the basis of the known ITS region, since extensive sequence data, even for very rare species, is already available for this region. This reliably ensures that all pathogenic dermatophytes, but not non-dermatophytes, are detected.

The identification of T. rubrum takes place indirectly by the specific amplification of the ESTRP gene of T. rubrum and T. violaceum, as well as the clear differentiation from T. violaceum with a universal primer pair, which is located in the ITS region and amplifies the genetic material of all dermatophytes (FIG. 1 and FIG. 3).

The differentiation of T. interdigitale occurs via a group-specific primer pair in the ESTRP gene that amplifies T. interdigitale and closely related species. Melting curve analysis provides a specific peak for T. interdigitale (FIG. 1 and FIG. 3).

Experimental Procedure:

Extraction of genomic DNA is performed using the QiaAmp DNA Mini Kit (QIAGEN) according to manufactures instructions with the following modifications: Proteinase K digestion is performed over night, after adding buffer ATL the mixture is incubated at 95° C. for 5 minutes. DNA is eluted in 45 μl AE buffer.

2 μl of DNA is used as template for PCR1 reaction, which contains 10 μl 2×HRM PCR Master Mix (Type-it HRM Kit (QIAGEN)), 0.7 μM of each primer (PCR1_for1 and PCR1_rev1, PCR1_for2 and PCR1_rev2, PCR1_for3 and PCR1_rev3) in a total volume of 20 μl per PCR reaction. Primers PCR1_for3 and PCR1_rev3 amplify a part of the ITS (internal transcribed spacer) region of the ribosomal DNA of any dermatophyte species. The other two primer pairs amplify two different regions of the ESTRP gene amplifying two species complexes. PCR1_for1 and PCR1_rev1 bind specifically T. tonsurans, T. equinum, T. interdigitale und T. mentagrophytes. PCR1_for2 and PCR1_rev2 amplify specifically T. rubrum, T. soudanese und T. violaceum.

High resolution melting analysis is performed using the LightCycler® 480 (ROCHE) with the following parameters. Initial Denaturation at 95° C. for 5 minutes is followed by 40 amplification cycles with 95° C. for 15 seconds and 60° C. for 40 sec. The temperature profile for the melting point detection is 95° C. for 1 min, 40° C. for 1 min, followed by continuous fluorescence data acquisition with 0.02° C./s (25 acquisitions per second). For melting point detection, the software LightCycler R 480 Software (release 1. 5. 0 SP4) (ROCHE) was used. Successful amplification of the initial PCR was assessed via the ‘Cp Fit Point method’ with the same software.

TABLE A
Primer sequences used for PCR1.

| | | SEQ | |
|---|---|---|---|
| name | Sequence 5′-3′ | ID NO | target |
| PCR1_for1 | ACTCCTCCAAACTACACCCAgCAA | 1 | ESTRP |
| PCR1_rev1 | TgCCCTCAgCAAgAgTTgCAAT | 3 | ESTRP |
| PCR1_for2 | AgCTCTgATCTCACCggTgACgATAg | 2 | ESTRP |
| PCR1_rev2 | gCTTggAgggCTggggAgT | 4 | ESTRP |
| PCR1_for3 | gCgCYCgCCRgAggA | 9 | ITS |
| PCR1_rev3 | CCggAACCAAgAgATCCgTTg | 10 | ITS |

For T. rubrum two melting peaks Tm1 at 80° C.−80.3° C. and Tm2 at 87.0° C.−87.5° C. will be detected. Specific for T. violaceum is a combination of Tm1=80.8° C.−81.3° C. and Tm2=86.9° C.−87.4° C. In these cases, the analysis is completed. If no melting peak at 79° C. or above is detected the sample is negative and the analysis is completed. If a melting peak between 79° C. and 81.5° C. is detected and/or a second peak below 88.8° C. the sample is dermatophyte positive and is not T. rubrum or T. violaceum.

In these cases, a second PCR reaction with HRM analysis using species specific Fluorescence Resonance Energy Transfer (FRET) probes is performed under the following conditions. PCR is performed with 2 μl template DNA in a 20 μl reaction volume with 0.5 U AmpliTaq® DNA Polymerase (Thermo Scientific™), 1× Buffer I (Thermo Scientific™), 0.2 mM dNTP Mix (BIOZYM), 0.12 μg BSA (NEB), each 0.4 μM of primers PCR2_for1, PCR2_for2, PCR2_for3, each 0.2 μM of primers PCR2_rev1, PCR2_rev2, PCR2_rev3 and 0.13 μM of each probe (Table B). Cycling conditions are 95° C. for 4 min, followed by 45 cycles with 95° C. for 10 sec, 56° C. for 20 sec and 72° C. for 20 sec.

Melting point detection follows with 95° C. for 1 min, 40° C. for 1 min, followed by continuous fluorescence data acquisition with 0.02° C./s. For melting point detection, the function ‘Tm calling’ and ‘HybProbe format’ are chosen using the software LightCycler R 480 Software (release 1. 5. 0 SP4) (ROCHE).

A unique melting peak will be obtained by at least one of the three labelled probes for the species T. tonsurans, T. equinum, T. interdigitale, T. mentagrophytes, T. simii, T. erinacei, T. benhamiae, T. concentricum, T. verrucosum, T. rubrum, T. violaceum, E. floccosum, M. canis, M. audouinii, M. ferrugineum and N. gypsea, N. fulva N. incurvata and N. persicolor. T. quinckeanum and T. schoenleinii will be detected as species complex.

TABLE B
primer and probe sequences used for PCR2

| | | SEQ ID | |
|---|---|---|---|
| name | 5′-3′ sequence | NO | target |
| PCR2_for1 | TCACTCTCgTCgTTgCTgCC | 5 | ESTRP |
| PCR2_rev1 | ggCAgCCgTggAggAgCA | 7 | ESTRP |
| PCR2_for2 | CCTAYggCCTCCTgATCgT | 6 | ESTRP |
| PCR2_rev2 | TggTggACTTTggTggCT | 8 | ESTRP |
| PCR2_for3 | CACATTAACTTggTCgTYAT | 11 | EF-1-α |
| | CggC | | |
| PCR2_rev3 | gAACTTCTCAATggTACg | 12 | EF-1-α |
| ESTRP I | gCCRCCgTCTCTgCCgTCAC | 13 | ESTRP |
| anchor | TCCTCCAAACTA-FL | | |
| ESTRP I | LC670-TCCCTgCCTCCCTC | 15 | ESTRP |
| probe | TggCAACCCAA-PH | | |
| ESTRP II | gAgggCACCggCgAgTACCA | 14 | ESTRP |
| anchor | gTACAgCA-FL | | |
| ESTRP II | LC610-CAAATTCggCgTCA | 16 | ESTRP |
| probe | AgAACgAgggCC-PH | | |
| EF-1-α | CgATACCACCgCACTTgTAg | 17 | EF-1-α |
| anker | ATCAAgTgACC-FL | | |
| EF-1-α | LC640-gTAATgTTATAgTC | 18 | EF-1-α |
| probe | AgTTTCTgTgTAATTCggT- | | |
| | PH | | |

### REFERENCES

1. Kraning K K, Odland G F. Prevalence, morbidity, and cost of dermatological diseases. J Invest Dermatol. 1979; 73(5 Pt 2):395-401.
2. Ghannoum M A, Hajjeh R A, Scher R, Konnikov N, Gupta A K, Summerbell R, et al. A large-scale North American study of fungal isolates from nails: the frequency of onychomycosis, fungal distribution, and antifungal susceptibility patterns. J Am Acad Dermatol. 2000 October; 43(4):641-8.
3. Haneke E, Roseeuw D. The scope of onychomycosis: epidemiology and clinical features. Int J Dermatol. 1999 Sep. 1; 38(S2):7-12.
4. Thomas J, Jacobson G A, Narkowicz C K, Peterson G M, Burnet H, Sharpe C. Toenail onychomycosis: an important global disease burden. J Clin Pharm Ther. 2010 October; 35(5):497-519.
5. Loo D S. Onychomycosis in the Elderly. Drugs Aging. 2007 Apr. 1; 24(4):293-302.
6. Salgo P L, Daniel C R, Gupta A K, Mozena J D, Joseph S W. Onychomycosis disease management. Med Crossfire Debates Peer Exch Insights Med. 2003; 4:1-17.
7. Hayutin A M. How population aging differs across countries: a briefing on global demographics. Stanf Cent Longev Retrieved August. 2007; 16:2009.
8. Sobera J, Elewski B. Onychomycosis. In: Nails: diagnosis, therapy and surgery. 3rd edition. Elsevier Saunders; 2005.
9. Tan J S, Joseph W S. Common Fungal Infections of the Feet in Patients with Diabetes Mellitus. Drugs Aging. 2004 Feb. 1; 21(2):101-12.
10. Weitzman I, Summerbell R C. The dermatophytes. Clin Microbiol Rev. 1995 April; 8(2):240-59.
11. Faergemann J, Baran R. Epidemiology, clinical presentation and diagnosis of onychomycosis. Br J Dermatol. 2003 Sep. 1; 149:1-4.
12. Effendy I, Lecha M, Feuilhade de Chauvin M, Di Chiacchio N, Baran R. Epidemiology and clinical classification of onychomycosis. J Eur Acad Dermatol Venereol. 2005 Sep. 1; 19:8-12.
13. Agathos M. Diagnose and Behandlung von Mykosen. Serie diabetisches Fu\s ssyndrom, Folge 6. MMW-Fortschr Med. 2000; (44).
14. Iii C R D, Elewski B E. The Diagnosis of Nail Fungus Infection Revisited. Arch Dermatol. 2000 Sep. 1; 136(9):1162-4.
15. Shemer A, Trau H, Davidovici B, Grunwald M H, Amichai B. Nagelproben bei Onychomykose:Vergleichsstudie mit einer Kurettage an drei Stellen des infizierten Nagels. JDDG J Dtsch Dermatol Ges. 2007 December; 5(12):no-no.
16. White T J. White, T. J., T. D. Bruns, S. B. Lee, and J. W. Taylor. Amplification and direct sequencing of fungal ribosomal RNA Genes for phylogenetics. 1990; 315-22.
17. Schmidt O. Indoor wood-decay basidiomycetes: damage, causal fungi, physiology, identification and characterization, prevention and control. Mycol Prog. 2007 Nov. 1; 6(4):261.
18. Ohst T, Kupsch C, Gräser Y. Detection of common dermatophytes in clinical specimens using a simple quantitative real-time TaqMan polymerase chain reaction assay. Br J Dermatol. 2015 Nov. 1; n/a-n/a.
19. Bergmans A M C, van der Ent M, Klaassen A, Böhm N, Andriesse G I, Wintermans R G F. Evaluation of a single-tube real-time PCR for detection and identification of 11 dermatophyte species in clinical material. Clin Microbiol Infect Off Publ Eur Soc Clin Microbiol Infect Dis. 2010 June; 16(6):704-10.
20. Beifuss B, Bezold G, Gottlöber P, Borelli C, Wagener J, Schaller M, et al. Direct detection of five common dermatophyte species in clinical samples using a rapid and sensitive 24-h PCR-ELISA technique open to protocol transfer. Mycoses. 2011 March; 54(2):137-45.
21. Brillowska-Dabrowska A, Saunte D M, Arendrup M C. Five-hour diagnosis of dermatophyte nail infections with specific detection of Trichophyton rubrum. J Clin Microbiol. 2007 April; 45(4):1200-4.
22. Reischl U, Drosten C, Gei\s sdörfer W, Gabel U, Hoffmann K S, Mauch H, et al. MiQ 1, Nukleinsäure-Amplifikationstechniken (NAT). Mikrobiol-Infekt Qual MiQ Münch Elsevier. 2011;
23. Brasch P D J. Bewährte und neue Verfahren zur Differenzierung von Dermatophyten. Hautarzt. 2004 Feb. 1; 55(2):136-42.
24. Nenoff P, Uhrlaß S, Krüger C, Erhard M, Hipler U-C, Seyfarth F, et al. Trichophyton species of Arthroderma benhamiae—a new infectious agent in dermatology. J Dtsch Dermatol Ges J Ger Soc Dermatol JDDG. 2014 July; 12(7):571-81.
25. Kupsch, C., Ohst, T., Pankewitz, F., Nenoff, P., Uhrlaß, S., Winter, I., et al. The agony of choice in dermatophyte diagnostics—performance of different molecular tests and culture in the detection of T. rubrum and T. interdigitale. Clin Microbiol Infect.
26. Winter I, Uhrlaß S, Kruger C, Herrmann J, Bezold G, Winter A, et al. Molekularbiologischer Direktnachweis von Dermatophyten im klinischen Material bei Verdacht auf Onychomykose und Tinea pedis. Hautarzt. 2013 Mar. 28; 64(4):283-9.
27. Mirhendi H, Makimura K, Hoog D, Sybren G, Rezaei-Matehkolaei A, Najafzadeh M J, et al. Translation elongation factor 1-α gene as a potential taxonomic and identification marker in dermatophytes. Med Mycol. 2015 Apr. 1; 53(3):215-24.

1-15. (canceled)

16. A method for identifying one or more dermatophytes or nucleic acids thereof, comprising carrying out a nucleic acid amplification reaction on a sample suspected of comprising one or more dermatophytes and/or nucleic acids thereof, wherein said reaction comprises primers that hybridize with a nucleic acid molecule encoding a dermatophyte extracellular serine/threonine-rich protein (ESTRP gene), and assessing the product of the amplification reaction.

17. The method of claim 16, wherein assessing of the product of the nucleic acid amplification reaction comprises a melting curve analysis.

18. The method of claim 17, comprising differentiating an identified dermatophyte from other dermatophyte species, wherein a unique melting temperature is assigned for the product of the nucleic acid amplification reaction for one or more of multiple dermatophyte species.

19. The method of claim 16, wherein the nucleic acid amplification reaction is a quantitative real-time polymerase chain reaction (qRT-PCR).

20. The method of claim 17, comprising a melting curve analysis using a sequence-unspecific double-stranded DNA binding dye, and/or one or more labelled sequence-specific probes that hybridizes to the ESTRP gene.

21. The method of claim 16, wherein the method comprises determining the presence of and/or differentiating between one or more species of the genera Trichophyton, Epidermophyton, Microsporum and/or Nannizzia.

22. The method of claim 16, wherein the primers that hybridize with the ESTRP gene are configured such that:
a. the ESTRP gene sequence bound by the primers exhibits two or fewer nucleotide differences in dermatophyte species within the genera Trichophyton, Epidermophyton and Microsporum, and/or species of the genus Nannizzia, enabling amplification of the ESTRP gene sequence of any one or more of said species, and
b. the ESTRP gene sequence between the sequences bound by the primers and amplified by the nucleic acid amplification reaction exhibits sufficient sequence diversion between one or more of dermatophyte species within the genera Trichophyton, Epidermophyton, Microsporum, and/or Nannizzia to enable unique melting temperatures in a melting curve analysis for one or more of said dermatophyte species and/or genera.

23. The method of to claim 22, wherein the ESTRP gene sequence between the sequences bound by the primers and amplified by the nucleic acid amplification reaction exhibits sufficient sequence diversion between one or more of dermatophyte species Microsporum canis, M. ferrugineum, Nannizzia gypsea, N. fulva, N. incurvata and N. persicolor to enable unique melting temperatures in a melting curve analysis for one or more of said dermatophyte species and/or genera.

24. The method of claim 16, comprising:
a. a first qRT-PCR reaction, wherein said first reaction comprises primers that hybridize with a dermatophyte ESTRP gene, and wherein the first reaction is assessed using a melting curve analysis with a sequence-unspecific double-stranded DNA binding dye, and
b. a second qRT-PCR reaction, wherein said second reaction comprises primers that hybridize with a dermatophyte ESTRP gene, and wherein the second reaction is assessed using a melting curve analysis with one or more labelled sequence-specific probes that hybridize to the ESTRP gene.

25. The method of claim 24, wherein the second reaction comprises a first and a second labelled sequence-specific probe that hybridize to the ESTRP gene, wherein
c. the first probe (anchor probe) is 15 to 40 nucleotides in length and hybridizes to a conserved sequence of the dermatophyte ESTRP gene with two or fewer nucleotide differences in the ESTRP gene sequence in dermatophyte species within the genera Trichophyton, Epidermophyton and Microsporum, and/or species of the genus Nannizzia, and
d. the second probe (species-specific probe) is 15 to 40 nucleotides in length and hybridizes to a sequence of the dermatophyte ESTRP gene with sufficient sequence diversion between one or more of the species within the genera Trichophyton, Epidermophyton, Microsporum, and/or Nannizzia to enable unique melting temperatures in a melting curve analysis for said second probe for one or more of said dermatophyte species and/or genera, and
e. said first and second probes hybridize in proximity to each other on the ESTRP gene and comprise labels enabling fluorescence resonance energy transfer (FRET) when in physical proximity.

26. The method of claim 25, wherein the first probe hybridizes to a conserved sequence of the dermatophyte ESTRP gene with two or fewer nucleotide differences in the ESTRP gene sequence in dermatophyte species within the genera Trichophyton, Epidermophyton and Microsporum, and species of Nannizzia gypsea, N. fulva and N. incurvata, and wherein the second probe hybridizes to a sequence of the dermatophyte ESTRP gene with sufficient sequence diversion between one or more of the species Microsporum canis, M. ferrugineum, Nannizzia gypsea, N. fulva, N. incurvata and N. persicolor to enable unique melting temperatures in a melting curve analysis for said second probe for one or more of said dermatophyte species and/or genera.

27. The method of claim 1, wherein the primers that hybridize with the ESTRP gene bind to and amplify a region of the ESTRP gene positioned between nucleotides 1 and 250 and/or between nucleotides 250 and 470 and/or between nucleotides 470 and 768, with reference to the ESTRP gene from T. verrucosum strain HKI 0517 according to SEQ ID NO 19.

28. The method of claim 27, wherein the primers for a first reaction comprise or consist of a sequence according to one or more of SEQ ID NO 1 and/or 2 as forward primers and SEQ ID NO 3 and/or 4 as reverse primers, and/or wherein the primers for a second reaction comprise or consist a sequence according to one or more of SEQ ID NO 5 and/or 6 as forward primers and SEQ ID NO 7 and/or 8 as reverse primers.

29. The method of claim 24, wherein the one or more sequence-specific probes of the second reaction bind to a region of the ESTRP gene between nucleotides 70 and 110 and/or between nucleotides 360 and 410, with reference to the ESTRP gene from T. verrucosum strain HKI 0517 according to SEQ ID NO 19.

30. The method of claim 29, wherein the probes comprise or consist of a sequence according to one or more of SEQ ID NO 13 and/or 14 as anchor probes and SEQ ID NO 15 and/or 16 as species specific probes.

31. The method of claim 16, wherein:
a. a first reaction comprises additionally primers that hybridize with a dermatophyte internal transcribed spacer region 1 and/or 2 (ITS1 and/or ITS2 region), and
b. a second reaction comprises additionally primers that hybridize with a dermatophyte translation elongation factor 1-α gene (EF-1-alpha gene).

32. The method of claim 31, wherein the primers that hybridize with the ITS1 and/or ITS2 region bind to and amplify a region of the ITS1 and/or ITS2 region between nucleotides 150 and 350, with reference to the ITS1 and/or ITS2 region from T. rubrum according to SEQ ID NO 27, and/or wherein the primers comprise or consist of a sequence according to SEQ ID NO 9 as forward primer and SEQ ID NO 10 as reverse primer, and/or
wherein the primers that hybridize with the EF-1-alpha gene bind to and amplify a region of the EF-1-alpha gene between nucleotides 1 and 230, with reference to the EF-1-alpha gene from T. rubrum according to SEQ ID NO 28, and/or wherein the primers comprise or consist of a sequence according to SEQ ID NO 11 as forward primer and SEQ ID NO 12 as reverse primer, and/or
wherein one or more sequence-specific probes are employed in the second reaction and bind to a region of the EF-1-alpha gene, and/or wherein the probes comprise or consist of a sequence according to one or more of SEQ ID NO 17 as anchor probe and SEQ ID NO 18 as species specific probe.

33. A kit for identifying one or more dermatophytes or nucleic acids thereof, comprising one or more reagents for a nucleic acid amplification reaction on a sample suspected of comprising one or more dermatophytes and/or nucleic acids thereof, wherein said reagents comprise (i) primers that hybridize with a ESTRP gene and (ii) software configured for identifying dermatophytes and/or differentiating dermatophytes from other dermatophyte species on the basis of unique melting temperatures assigned to the products of the nucleic acid amplification reaction for one or more multiple dermatophyte species.

34. The kit of claim 33, comprising additionally:
a. One or more sequence-unspecific double-stranded DNA binding dyes, and
b. One or more labelled sequence-specific probes that hybridize to a dermatophyte ESTRP gene.

35. An isolated oligonucleotide of 15 to 40 nucleotides in length, comprising or consisting of a sequence according to any one of:
a. SEQ ID NO 1-8, in the form of a primer; or
b. SEQ ID NO 13-16, in the form of a probe.
